SEQUENCE LISTING (19 sequences)

SEQ ID NO: 1 - protein, 5 aa, Synthetic construct
XGGGC

SEQ ID NO: 2 - protein, 12 aa, synthetic construct
TSTWNLYVPPEP

SEQ ID NO: 3 - protein, 12 aa, synthetic construct
WPGPARTQYHRY

SEQ ID NO: 4 - protein, 12 aa, synthetic construct
WPGPARTQYHAY

SEQ ID NO: 5 - protein, 10 aa, Synthetic construct
RMYQSLLAPS

SEQ ID NO: 6 - protein, 12 aa, Synthetic construct
YSSATFRLHNIS

SEQ ID NO: 7 - protein, 12 aa, Synthetic construct
SYALSSASVRTK

SEQ ID NO: 8 - protein, 16 aa, Synthetic construct
TSTWNLYVPPEPGGGC

SEQ ID NO: 9 - protein, 14 aa, Synthetic construct
RMYQSLLAPSGGGC

SEQ ID NO: 10 - protein, 16 aa, Synthetic construct
YSSATFRLHNISGGGC

SEQ ID NO: 11 - protein, 16 aa, Synthetic construct
SYALSSASVRTKGGGC

SEQ ID NO: 12 - protein, 16 aa, Synthetic construct
WPGPARTQYHRYGGGC

SEQ ID NO: 13 - protein, 16 aa, Synthetic construct
WPGPARTQYHAYGGGC

SEQ ID NO: 14 - protein, 16 aa, Synthetic construct
CGGGTSTWNLYVPPEP

SEQ ID NO: 15 - protein, 14 aa, Synthetic construct
CGGGRMYQSLLAPS

SEQ ID NO: 16 - protein, 16 aa, Synthetic construct
CGGGYSSATFRLHNIS

SEQ ID NO: 17 - protein, 16 aa, Synthetic construct
CGGGSYALSSASVRTK

SEQ ID NO: 18 - protein, 16 aa, Synthetic construct
CGGGWPGPARTQYHRY

SEQ ID NO: 19 - protein, 16 aa, Synthetic construct
CGGGWPGPARTQYHAY

(19) United States
(12) Patent Application Publication
(10) Pub. No.: US 2026/0347905 A1
(43) Pub. Date: Nov. 5, 2026

(54) IMPEDIMETRIC DETECTION USING PEPTIDE AND PEPTIDE MIXTURES

(71) Applicant: Yissum Research Development Company of the Hebrew University of Jerusalem Ltd., Jerusalem (IL)

(72) Inventors: Zvi HAYOUKA, Mazkeret Batya (IL); Mattan HUREVICH, Jerusalem (IL); Shlomo YITZCHAIK, Jerusalem (IL); Tal STERN BAUER, Jerusalem (IL)

(73) Assignee: Yissum Research Development Company of the Hebrew University of Jerusalem Ltd., Jerusalem (IL)

(21) Appl. No.: 19/106,293

(22) PCT Filed: Aug. 17, 2023
(86) PCT No.: PCT/IL2023/050871

§ 371 Date: Feb. 25, 2025

Related U.S. Application Data
(60) Provisional application No. 63/428,962, filed on Nov. 30, 2022.

(51) Int. Cl. C12Q 1/22; C07K 7/06; C07K 7/08; G01N 27/02; G01N 27/36

(52) U.S. Cl. CPC C12Q 1/22; C07K 7/06; C07K 7/08; G01N 27/026; G01N 27/36

(57) ABSTRACT

The technology disclosed herein concerns an impedimetric method for determining presence and quantity of microorganisms in a sample.

### FIELD OF THE INVENTION

The technology disclosed herein generally concerns devices and impedimetric methods for determining microorganism populations.

### BACKGROUND OF THE INVENTION

Electrochemical biosensors are integrated devices that convert specific biological interactions into electrical output that provides analytical information, hence are suitable for bacteria detection. They can be fabricated at a relatively low cost and can provide a rapid and accurate analysis. Electrochemical impedance spectroscopy (EIS) is a powerful technique used for the analysis of interfacial properties related to bio-recognition events occurring at the electrode surface. Faradaic impedance measurement is performed in the presence of redox species. The changes in resistance to charge transfer (R_{CT}) translate the molecular recognition event on the monolayer to a measurable signal. Thus, EIS enables label-free detection of biomolecules and biochemical interactions.

Monolayers composed of aptamers, antibodies, polymers, and peptides are used for bacterial detection. Peptides are easy to synthetize and modify and can be tailored rather simply to surface applications. Thiol-Au is the simplest and most common strategy to assemble peptide monolayers. The process of self-assembled monolayer (SAM) organization includes an initial stage of fast thiol-gold bond formation. The second stage is a slow process of organization where the immobilized molecules assemble to maximize the intermolecular interactions between them. While interactions between the peptides might stabilize the monolayer and lead to high density, repulsion due to electrostatic and/or steric hindrance can hinder monolayer assembly.

Antimicrobial peptides (AMPs) are short natural or synthetic peptides that eradicate bacteria through interactions with their membrane. AMPs are highly diverse in sequences, lengths and structures. However, there are two common features of AMPs: cationic charge and high degree of hydrophobicity. Random peptide mixtures (RPMs) are peptides that use the advantage of AMPs common features. RPMs are composed of 20 amino acids chain lengths that combine hydrophobic and cationic amino acid residues in random sequences. RPMs are easy to synthesize in large quantities (FIG. 1). Previous studies revealed that RPMs have broad-spectrum antimicrobial activity. Their proposed mechanism of action involves electrostatic and hydrophobic interactions with the negatively charged bacterial membrane via pore formation or non-selective distribution on the membrane. In addition, RPMs immobilized on polystyrene beads bind and eliminate bacterial cells very efficiently. These previous studies suggested that RPMs could be used as recognition molecules for electrochemical sensing of bacteria.

Unlike assembly of homogeneous peptides on gold that are expected to self-organize and to form strong intermolecular interactions, the assembly of RPMs has not been explored yet. RPMs contain mixtures of entities with different sequences and distribution of positive charge/hydrophobic residues along the chain length that may affect the assembly.

### SUMMARY OF THE INVENTION

The present invention concerns a sensor unit in a form of a substrate functionalized with a recognition peptide or a mixture of recognition peptides such as a random peptide mixture (RPM) that are capable of binding or interacting with a microorganism present in a sample. The functionalized substrate may be used for qualitatively and/or quantitatively determine presence of a microorganism (presence and/or amount), and/or for determining a taxonomical type of the microorganism, and/or for determining viability of a microorganism in a liquid medium in which the microorganism is present or suspected of being present. The sensor unit implements a recognition monolayer of one or more recognition peptides having an ability to or capable of interacting with microorganisms, such as bacteria, in a liquid medium. Surprisingly, and as demonstrated below, detection of the microorganisms was possible despite the immobilization of the recognition peptides to the surface and the inherent spatial restriction on their ability to fold or form 3D structures with the target microorganism. The superiority of sensors of the invention has been demonstrated by the sensor's ability to detect presence, amount, specificity (type) and viability of a variety of microorganisms by utilizing electrochemical techniques such as electrochemical impedance spectroscopy (EIS) and cyclic voltammetry.

Thus, in a first of its aspects, there is provided a recognition surface associated with a monolayer of recognition peptide molecules capable of interacting with a microorganism in a liquid medium.

The invention further provides a recognition surface for use in electrochemical impedance spectroscopy (EIS), the surface being associated with a unidirectional or a multidirectional layer of recognition peptide molecules capable of interacting with a microorganism present in a liquid medium (or present in a solid surface, in an aerosol or a in gas sample which may be sampled into a liquid medium or measured directly), wherein said interaction is detectable by EIS.

Further provided is an electrode for use in electrochemical impedance spectroscopy (EIS), the electrode having a surface associated with a monolayer of recognition peptide molecules capable of interacting with a microorganism present in a liquid medium, in an aerosol or in a gas phase (or collected from such media).

The “recognition surface” of the invention, as defined herein, or as implemented on an electrode, a sensor or any device, is a surface associated with a layer of at least one recognition peptide capable of interacting with a microorganism. The layer is typically a monolayer of a single type of recognition peptide or a mixture of recognition peptides, wherein each peptide in the layer is oriented in a direction away from the surface. In some cases, the orientation away from the surface is in a direction perpendicular to or substantially perpendicular to the surface. Such an orientation, wherein all or a majority of the recognition peptides in the layer are directed away from the surface, is regarded herein as “unidirectional” or “unidirectionally oriented”. However, in some cases, the orientation of the peptides is random and “multidirectional orientation” may be observed.

The surface material or the electrode material onto which a layer or a film of recognition peptide(s) is formed, is selected or structured to provide selective sensing of an interaction between a microorganism and the recognition peptide, wherein the interaction may be detectable, e.g., by EIS. Without wishing to be bound by theory, in order to achieve a robust association or immobilization of the recognition peptide molecules onto the surface, or a region thereof, the association or immobilization typically involves chemical adsorption (or chemisorption) via surface binding groups which may be part of the peptide structure or may be substituted thereon before or during the deposition process. The surface binding groups may be amines, carboxylic acids, thiols, disulfides or generally sulfur-containing functionalities, phosphates or generally phosphorus-containing functionalities, and others, as disclosed herein, and as known in the art. Surface association or immobilization may form a monolayer of recognition peptides on a surface region or the complete surface. The monolayer may be homogenous in composition, namely comprising a single type of peptides; or may be heterogeneous, namely comprising peptides of different compositions. In some cases, the monolayer may be formed of a mixture of different recognition peptide populations or types, wherein each population or type differs in composition from the other. Irrespective of the population of peptides, a monolayer formed is unidirectional, or multidirectional, as defined herein.

The recognition peptide structure may comprise a surface binding group or may be chemically modified to include such a group. The surface binding group is selected based on the nature of the so-called surface to which the recognition peptide is to be associated or onto which it is to be immobilized. Generally speaking, the recognition surface may be made of any sensor material as known in the art, and may be provided in any shape and form. In some cases, the surface is of a conductive material. Non-limiting examples of surfaces used include gold surfaces and glassy carbon electrode (GCE) substrates.

The surface binding group allowing direct immobilization of the recognition peptide onto the surface may be an atom or a group of atoms having affinity to the surface material. In some embodiments, the surface binding atom or group of atoms may be hydroxy (OH), thiol (SH), disulfide (—S—S—), azide (—N₃), amine (—NH₂, or a primary, secondary or tertiary amine), carboxy (COO), phosphate groups and others.

In some embodiments, the recognition peptide is substituted by or includes at least one surface-binding group selected from hydroxy (OH), thiol (SH), disulfide (—S—S—), azide (—N₃), amine (—NH₂, or a primary, secondary or tertiary amine), carboxy (COO), and a phosphate group.

In some embodiments, the recognition peptide is substituted by or includes at least one surface-binding group selected from thiol (SH), disulfide (—S—S—), amine (—NH₂, or a primary, secondary or tertiary amine) and carboxy (COO).

In some embodiments, the recognition peptide is substituted by or includes a sulfur containing group, e.g., a thiol or a disulfide group.

In some embodiments, the recognition peptide comprises a sulfur containing group, e.g., a thiol or a disulfide group, or a sulfur atom through which surface association may be achievable.

In some embodiments, the surface binding group is an amino acid that is selected based on the presence of a functionality capable of surface association. In some embodiments, the surface binding group comprises a sulfur atom, e.g., methionine, cysteine, homocysteine and taurine. In some embodiments, the surface binding group is cysteine.

In some cases, the recognition peptide is functionalized with a linker group that links or bridges the recognition peptide and the surface binding group. In some embodiments, the linker group is or comprises an amino acid or a short amino acid sequence (a short peptide being a 2 to 5-amino acid peptide of same or different amino acids). The amino acid or short peptide linker is different from the recognition peptide, yet may comprise any amino acid as known in the art. For example, the amino acid may be alanine, arginine, asparagine, aspartic acid, cysteine, glutamine, glutamic acid, glycine, and others, as known in the art.

In some embodiments, the recognition peptide is functionalized with a linker group consisting or comprising one or more amino acids, typically between 1 and 5 amino acids, each selected as herein. In some embodiments, the linker group is glycine (G, Gly) and/or cysteine (C, Cys) or comprises glycine and/or cysteine. In some embodiments, the linker group is or comprises Gly and Cys.

In some embodiments, the linker group is a tripeptide, a tetrapeptide or a pentapeptide, optionally comprising Gly and/or Cys.

In some embodiments, the linker group is a tetrapeptide comprising Gly and Cys. In some embodiments, the tetrapeptide is -Gly-Gly-Gly-Cys (-GGGC).

In a sequence such as -Gly-Gly-Gly-Cys (-GGGC), the “-” designates connectivity to the recognition peptide and Cys is the surface binding group.

Whether the surface binding group is directly associated to the recognition peptide or is associated thereto via a linker group, as disclosed, the surface binding group or the linker group may be substituted on the C-terminal or the N-terminal of the recognition peptide, or through any side chain or group. The site of substitution may vary.

In some embodiments, the recognition peptide is immobilized onto a gold surface via a thiol-containing group. In some embodiments, the recognition peptide comprises a C-terminal or a N-terminal linker, or a side chain linker of the form, e.g., Gly-Gly-Gly-Cys (-GGGC).

Thus, the invention provides a gold recognition surface associated with a layer of recognition peptide molecules, optionally unidirectional, of the form PP-L-Cys, wherein PP is a recognition peptide capable of interacting with a microorganism, L is a linker group, as defined herein, which may or may not be present, and wherein Cys is a surface binding group.

The invention also provides a gold recognition surface associated with a layer of recognition peptide molecules of the form PP-Gly-Gly-Gly-Cys (PP-GGGC), wherein PP is a recognition peptide capable of interacting with a microorganism, Gly and Cys are glycine and cysteine, respectively, and wherein Cys is a surface binding group.

The invention further provides a recognition surface for use in electrochemical impedance spectroscopy (EIS), the surface being a gold surface that is associated with a layer of recognition peptide molecules of the form PP-Gly-Gly-Gly-Cys (PP-GGGC), wherein PP is a recognition peptide capable of interacting with a microorganism in a liquid medium, Cys is a surface binding group and wherein said interaction is detectable by EIS.

Further provided is an electrode for use in electrochemical impedance spectroscopy (EIS), the electrode having a gold surface associated with a layer of recognition peptide molecules of the form PP-Gly-Gly-Gly-Cys (PP-GGGC, SEQ ID NO 1), the association is through the Cys group and wherein PP is a recognition peptide capable of interacting with a microorganism in a liquid medium.

The “recognition peptide” (designated PP in the structures above) is any peptide or a mixture of peptides that is/are known or determined to be capable of associating or interacting with a microorganism. The recognition peptide may be selected based on the microorganism to be detected or evaluated or may be a generic peptide having a degree of interaction with a microorganism that is nonselective. Several assay methods are known to determine an interaction or association of a peptide to a microorganism. Any of these methods may be used to determine in advance the suitability of any given peptide, or a mixture of peptides, as a recognition peptide(s). The methods include labeling methods, flow cytometry (FC), surface plasmon resonance (SPR), quartz crystal microbalance (QCM) techniques, dye-based microscopy and others.

In some cases, flow cytometry (FC) may be used.

As known in the art, flow cytometry (FC) is a technique used to detect and measure physical and chemical characteristics of a population of cells or particles in a suspension. To identify suitable recognition peptides, FC was used to measure the binding of a labeled peptide to cells of a target microorganism, such as bacterial cells. In an exemplary non-limiting set up, various peptides were labeled with a 5,6-carboxyfluorescein at the N′ terminus. Various bacterial cells, e.g., MRSA, E. coli RP and PAO1 bacterial cells, were incubated with the labeled peptides. After several cycles of washing, the bacterial cells were re-suspended in fresh PBS. The samples were filtered and analyzed by the FC system. FC results were analyzed to quantify the labeled bacterial cells compared to the non-labeled to estimate the ability of any of the labeled peptides to bind the target bacteria. Peptides which were found to bind or associate to the target microorganism were identified as ‘recognition peptides’ for the microorganism tested.

As a person versed in the art would appreciate, other methods may be used for determining interactions between a peptide and a microorganism.

As used herein, the terms “recognition”, “association”, “interaction” or any lingual variation thereof, when made in the context of a peptide and a microorganism, encompasses a characteristic ability of a peptide to undergo physical or chemical binding to cells of the microorganism. The binding may be reversible or irreversible; yet should exist for a period of time sufficient to be detected by methods of the invention, as disclosed herein.

The recognition peptides that are immobilized on a surface (to provide the recognition surface of the invention) may interact with the microorganism via any one group thereof (which may or may not be a most exposed group in a monolayer of the peptides) or the interaction may involve several groups, or the peptide as a whole. As the mechanism and site of interaction is unimportant for purposes of determining presence, amount, type and vitality of a target microorganism, to permit interaction through any part of the recognition peptide, the monolayer of recognition peptides formed on the surface should not be dense. Typically, monolayers of the recognition peptide are characterized by a surface coverage that is between 20 and 80%, with no apparent distinct domains of regions that are of different layer density. In other words, the monolayers are generally homogenous.

In some embodiments, monolayers of a recognition peptide or a mixture of such peptides exhibit between 20 and 80% surface coverage or between 20 and 70%, 20 and 60%, 20 and 50%, 20 and 40%, 20 and 30%, 30 and 70%, 40 and 70%, 50 and 70%, 60 and 70%, 30 and 50%, or between 40 and 60% surface coverage.

The recognition peptide may be a single peptide or a mixture of different types of peptides, wherein each type differs from another type in structure, length, specific groups capable of association or interacting with the microorganism, specific surface-binding groups and so on. Irrespective of the peptide used and the plurality of different peptides, all or substantially all peptides are recognition peptides, as defined herein. For different reasons, it may be desired to provide a monolayer of different molecules, wherein a portion of the monolayer is composed of recognition peptides while other molecules in the monolayer may be used as spacers or naïve molecules that exhibit no interaction with microorganisms or are neutral to the presence of a microorganism.

In some embodiments, a monolayer formed and utilized as disclosed herein consists a single type of peptides (all peptides in the monolayer are the same). In other embodiments, the monolayer comprises a population of different peptides. In some embodiments, a monolayer comprises a plurality of surface-bound molecules, selected from peptide and non-peptide molecules, wherein the peptide molecules are recognition peptides as selected herein. The non-peptide molecules may be aliphatic molecules or others.

The recognition peptide is different from any of the peptide linkers which may be used to associate or bridge a recognition peptide and a surface binding group. For example, while the linker group may be a peptide comprising between 1 and 5 amino acids, which may be same or different, the recognition peptide is a longer peptide comprising between 8 and 50 amino acids. Thus, a recognition peptide used according to the invention may be of the general structure PP-L-X, wherein PP is a recognition peptide, L is a linker group which may or may not be present and which may be an amino acid or an amino acid sequence (a short peptide, as disclosed herein) and wherein X is a surface binding group or atom. In some cases, both the linker and the surface binding group are part of the recognition peptide, and in some other cases, one or both of the linker and surface binding groups are substituted, e.g., onto the C- or N-terminal of the recognition peptide.

As disclosed herein, the term “peptide”, in general, refers to a peptide molecule, as known in the art, that comprises or consists an amino acid sequence which length depends on the particular peptide used. Typically, all peptides used according to the invention for constructing the recognition surfaces, sensors and other devices, are recognition peptides. In some cases, the recognition peptides may be associated to or provided in combination with peptides that are not recognition peptides. Where a monolayer of recognition peptides is formed of a single type of recognition peptide (a homogenous monolayer), the recognition peptide may comprise between 8 and 50 amino acids. In case the monolayer is formed of a mixture of recognition peptides, e.g., a random mixture of recognition peptides, RPM, each peptide in the mixture may comprise between 4 and 20, or 4 and 15 amino acids.

The amino acids making up a recognition peptide used according to the invention may be selected amongst the known amino acids, or amongst hydrophobic and cationic amino acids. The number of amino acids of any type may vary; yet in some cases is random. Each of the amino acids in recognition peptides disclosed herein may be associated to another amino acid via a peptide bond. In some embodiments, the amino acids making up a recognition peptide, are all hydrophobic amino acids or all cationic amino acids. In other words, in such embodiments, the recognition peptide consists of hydrophobic amino acids or cationic amino acids. In other embodiments, the recognition peptide may comprise a random combination of hydrophobic amino acids and cationic amino acids.

The recognition peptides may comprise L-amino acids, D-amino acids (e.g., D-Lys), or combinations thereof. The amino acids may be selected from natural and non-natural amino acids. Recognition peptides having both D-amino acid residues and L-amino acid residues are defined herein as heterochiral peptides. In some embodiments, the recognition peptide comprises all L-amino acids. In some other embodiments, the recognition peptide comprises all D-amino acid. In further embodiments, the recognition peptide may be selected amongst heterochiral peptides.

Where a recognition peptide is a random peptide mixture, RPM, the selection of amino acids may dictate the number of recognition peptides in the mixture. For example, the number of recognition peptides in a mixture may be at least 2ⁿ (wherein n is the length of the peptide), where two amino acids are used. The number of peptides may be 3ⁿ in cases where one of the two amino acids is in the D- or L-form, and at least 4ⁿ in cases where both amino acids are in the L- and D-forms.

In some embodiments, a recognition peptide used alone or in an RPM combination may be or may comprise natural or non-natural amino acids. In some embodiments, an RPM of the invention further comprises recognition peptides constructed of non-natural amino acids.

In some embodiments, a recognition peptide may comprise amino acid(s) selected from β-amino acids, γ-amino acid, D-amino acid, cyclic peptides and any combination thereof.

In some embodiments, a recognition peptide may be a cyclic peptide.

In some embodiments, a recognition peptide used according to the invention is a glycosylated peptide or an acetylated peptide.

In some embodiments, the number of amino acid residues in a recognition peptide is at least 4 but no more than 50 or no more than 30 or no more than 20, inclusive. In some embodiments, the number of amino acids is between 4 and 15. In some embodiments, the number of amino acid residues is 5, or 6, or 7, or 8, or 9, or 10, or 11, or 12, or 13, or 14, 15, 16, 17, 19 or 20.

In some embodiments, the recognition peptide comprises at least five amino acid residues. In some embodiments, the recognition peptide consists of between 5 and 10 amino acid residues, 5 and 8 amino acid residues or 5 and 7 amino acid residues. In some embodiments, the recognition peptide consists of between 15 and 30 amino acid residues, 15 and 25 amino acid residues or 20 and 17 amino acid residues. In some embodiments, the number of amino acids in a recognition peptide used according to the invention is 7, 8, 9, 10, 11, 12, 13, 14 or 15.

The number of hydrophobic amino acids relative to the number of cationic amino acids (namely the ratio of the two), as well as the proximity of the hydrophobic amino acid to the fatty moiety, may be selected as needed.

In some embodiments, the ratio between cationic and hydrophobic amino acids (C:H) is at least 1:1. In some embodiments, the ratio C:H is at least 2:1. In some embodiments, the ratio is at least 3:1. In some embodiments, the ratio is at least 4:1. In some embodiments, the ratio is at least 5:1. In some embodiments, the ratio is at least 6:1. In some embodiments, the ratio is at least 7:1. In some embodiments, the ratio is at least 8:1. In some embodiments, the ratio is at least 9:1.

In some embodiments, the ratio C:H is between 1:1 and 10:1.

The “cationic amino acids” may be selected from amino acids having positively charged side chains, as known in the art, or any derivative thereof. Non-limiting examples include lysine, arginine, histidine, ornithine, di-amino butyric acid (Dab) and di amino propionic acid (Dap). In some embodiments, the cationic amino acid is selected from lysine, arginine and histidine. In some embodiments, the cationic amino acid is lysine or arginine.

The “hydrophobic amino acids” may be selected from the amino acids having low or no water solubility, or any derivative thereof. Non-limiting examples include proline, methionine, tryptophan, phenylalanine, leucine, isoleucine, glycine, alanine and valine. In some embodiments, the hydrophobic amino acid is selected from phenylalanine, leucine, isoleucine, glycine, alanine and valine. In some embodiments, the hydrophobic amino acid is phenylalanine.

In some embodiments, the recognition peptide is a single type of peptide and a monolayer implemented in a recognition surface is formed thereof.

In some embodiments, the recognition peptide forms a homogenously dense monolayer on a region of a recognition surface.

In some embodiments, a monolayer is provided to comprise a homogeneous or a mixed population of recognition peptide, wherein at least one of the recognition peptides having a sequence selected from:

1.
(SEQ ID NO 2)
TSTWNLYVPPEP
2.
(SEQ ID NO 3)
WPGPARTQYHRY
3.
(SEQ ID NO 4)
WPGPARTQYHAY
4.
(SEQ ID NO 5)
RMYQSLLAPS
5.
(SEQ ID NO 6)
YSSATFRLHNIS,
and
6.
(SEQ ID NO 7)
SYALSSASVRTK.

In some embodiments, the recognition peptide is:

1.
(SEQ ID NO 2)
TSTWNLYVPPEP,
or
2.
(SEQ ID NO 3)
WPGPARTQYHRY,
or
3.
(SEQ ID NO 4)
WPGPARTQYHAY,
or
4.
(SEQ ID NO 5)
RMYQSLLAPS,
or
5.
(SEQ ID NO 6)
YSSATFRLHNIS,
or
6.
(SEQ ID NO 7)
SYALSSASVRTK.

In some embodiments, the recognition peptide is:

1.
(SEQ ID NO 2)
TSTWNLYVPPEP,
or
2.
(SEQ ID NO 3)
WPGPARTQYHRY,
or
3.
(SEQ ID NO 4)
WPGPARTQYHAY.

In some embodiments, a monolayer is formed of a recognition peptide having the sequence TSTWNLYVPPEP (SEQ ID NO 2), or a monolayer is formed of a recognition peptide having the sequence WPGPARTQYHRY (SEQ ID NO 3), or a monolayer is formed of a recognition peptide having the sequence WPGPARTQYHAY (SEQ ID NO 4), or a monolayer is formed of a recognition peptide having the sequence RMYQSLLAPS (SEQ ID NO 5), or a monolayer is formed of a recognition peptide having the sequence YSSATFRLHNIS (SEQ ID NO 6), or a monolayer is formed of a recognition peptide having the sequence SYALSSASVRTK (SEQ ID NO 7).

In some embodiments, the recognition peptide is functionalized with a linker group consisting or comprising one or more amino acids, typically between 1 and 5 amino acids. In some embodiments, the recognition peptide is selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4).

In some embodiments, the linker group is glycine (G, Gly) and/or cysteine (C, Cys) or comprises glycine and/or cysteine. In some embodiments, the linker group is or comprises Gly and Cys. In some embodiments, the linker group is a tripeptide, a tetrapeptide or a pentapeptide, optionally comprising Gly and/or Cys.

In some embodiments, the recognition peptide comprises one of TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4) and is associated to a tetrapeptide of the form-Gly-Gly-Gly-Cys (-GGGC), wherein Gly (G) is a surface binding group.

Each of the specific peptides disclosed herein is provided as a sequence of amino acids identified by their letter abbreviations, as known in the art. In these sequences, A=alanine, R=arginine, N=asparagine, D-aspartic acid, C-cysteine, Q-glutamine, E=glutamic acid, G-glycine, H=histidine, L-leucine, K=lysine, M=methionine, F=phenylalanine, P=proline, O-pyrrolysine, S=serine, U=selenocysteine, T-threonine, W-tryptophan, Y-tyrosine and V-valine.

In some embodiments, the recognition peptide is one of TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4) that is C- or N-associated to -Gly-Gly-Gly-Cys (-GGGC). Thus, the recognition peptide may be selected from

(SEQ ID NO 8)
TSTWNLYVPPEPGGGC,
(SEQ ID NO 9)
RMYQSLLAPSGGGC,
(SEQ ID NO 10)
YSSATFRLHNISGGGC,
(SEQ ID NO 11)
SYALSSASVRTKGGGC,
(SEQ ID NO 12)
WPGPARTQYHRYGGGC,
(SEQ ID NO 13)
WPGPARTQYHAYGGGC,
(SEQ ID NO 14)
CGGGTSTWNLYVPPEP,
(SEQ ID NO 15)
CGGGRMYQSLLAPS,
(SEQ ID NO 16)
CGGGYSSATFRLHNIS,
(SEQ ID NO 17)
CGGGSYALSSASVRTK,
(SEQ ID NO 18)
CGGGWPGPARTQYHRY,
and
(SEQ ID NO 19)
CGGGWPGPARTQYHAY,

wherein in each of the aforementioned peptides, the Cys group is optionally a surface binding group.

In some embodiments, the group-Gly-Gly-Gly-Cys (-GGGC) may be associated to the recognition peptide through any of the peptide side chains or non-terminal amino acids.

In some embodiments, any of the single peptides disclosed herein may be or covalently associated to fatty moieties (e.g., derived from fatty acids) or glycans, as defined and selected herein. The association may be through the C- or N-terminal or via any of the peptide side chains or non-terminal amino acids.

In some embodiments, the single peptides may be provided in combination with one or more other peptides.

In some embodiments, the recognition peptide is a random peptide mixture (RPM).

The term “random peptide mixture” or “RPM” refers to a mixture of two or more types of peptides. The mixture typically comprises two types of peptides:
recognition peptides having between 5 and 20 amino acids that are synthesized by mixing a defined ratio of a hydrophobic amino acid and a cationic amino acid, optionally also their enantiomers or other non-natural amino acids, to generate a mixture of peptides having desired chain lengths and having different peptide sequences and optionally different stereochemistries. These different sequences confer the ability to bind a wide variety of bacterial species, fungi and viruses; and
conjugates of recognition peptides having between 5 and 50 amino acids that are covalently associated to fatty moieties (e.g., derived from fatty acids) or glycans.

Each of the recognition peptides making up a monolayer of RPM is as defined and disclosed herein.

In some embodiments, recognition peptides of the RPM may comprise at least five amino acid residues. In some embodiments, the peptides consist of between 5 and 10 amino acid residues, 5 and 8 amino acid residues or 5 and 7 amino acid residues. In some embodiments, the peptides consist of between 15 and 30 amino acid residues, 15 and 25 amino acid residues or 20 and 17 amino acid residues. In some embodiments, the number of amino acids in peptides making up an RPM used according to the invention is 7, 8, 9, 10, 11, 12, 13, 14 or 15.

The number of hydrophobic amino acids relative to the number of cationic amino acids (namely the ratio of the two), as well as the proximity of the hydrophobic amino acid to the fatty moiety, can be modified as needed. In some embodiments, the ratio between cationic and hydrophobic amino acids (C:H) is at least 1:1. In some embodiments, the ratio C:H is at least 2:1. In some embodiments, the ratio is at least 3:1. In some embodiments, the ratio is at least 4:1. In some embodiments, the ratio is at least 5:1. In some embodiments, the ratio is at least 6:1. In some embodiments, the ratio is at least 7:1. In some embodiments, the ratio is at least 8:1. In some embodiments, the ratio is at least 9:1.

In some embodiments, the ratio C:H is between 1:1 and 10:1.

In some embodiments, the recognition peptides of an RPM comprise or consist of the amino acids phenylalanine and lysine. In some embodiments, in an ROM mixture, the peptides are constructed of identical amino acids (e.g., KKKKK). In some other embodiments, the peptides are random peptides, wherein each peptide has a sequence of amino acids that is different (e.g., some are KFKKK, some KKKKK, others are KKFFK, etc).

Additionally, in some embodiments where recognition peptides in mixtures of the invention are conjugated to fatty moieties, the fatty moieties are conjugated or associated to the peptide at a position that is further apart (e.g., at a maximal distance) from the at least one hydrophobic amino acid in the peptide(s).

In some embodiments, in a mixture of the invention, recognition peptides of 4 to 15 amino acids in length that are optionally conjugated to a fatty moiety, are presented, wherein each of the peptides in the mixture consists of hydrophobic and/or cationic amino acids, wherein the ratio, in the mixture, of total hydrophobic amino acids to total cationic amino acids is between 3:1 and 1:3. In some embodiments, the ratio in a mixture of the total hydrophobic amino acids to the total cationic amino acids is between 2:1 and 1:2. In some embodiments, the ratio is about 1:1.

In some embodiments, in a mixture of the invention, the recognition peptide consists of phenylalanine and/or lysine residues, wherein the ratio, in the mixture, of the total phenylalanine residues to the total lysine residues is between 3:1 and 1:3.

As used herein, the term “ratio”, as applied to the ratio amounts of amino acids within a mixture, refers to the ratio between different amino acid types, wherein each of the amino acid forms part of the peptides present in the mixture. For example, “a ratio in the mixture of the total hydrophobic amino acids to the cationic amino acids” refers to the number of all hydrophobic amino acids forming part of the peptides in the mixture relative to the number of all cationic amino acids forming part of the peptides in the same mixture.

As stated herein, recognition peptides used in RPM mixtures of the invention may be conjugated or covalently associated to a fatty moiety that may be derived from a fatty acid. Such a conjugation results in a lipophilic conjugate which properties may be varied and tailored by, e.g., a particular site of conjugation, a particular amino acid for conjugation, by selecting a suitable fatty acid, etc. The fatty moiety may be derived from saturated, unsaturated, monounsaturated and polyunsaturated fatty acids, as known in the art. The fatty moiety may consist of at least eight carbon atoms, and thus may be selected and derived from such fatty acids as decanoic acid, undecanoic acid, dodecanoic acid (lauric acid), myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, cerebronic acid and others.

The fatty moiety may be coupled to the N-terminal, to the C-terminal, or to any other free functional group along the peptide chain. As such, the moiety may be derived from any precursor molecule that upon reaction therewith results in covalent association of the fatty moiety with the atom or group on the peptide chain. The fatty moiety may thus be associated to the peptide (or an amino acid of the peptide) via any covalent functionality such as via an ester group, an amide group, an amine group, an oxo group, a thio group, and others.

In some embodiments, the fatty moiety is derived from palmitic acid or lauryl acid.

In some embodiments, an RPM mixture of the invention is a mixture or a combination of recognition peptides which comprises two or more peptides that have the same length (in terms of number of amino acids in the peptide) but differ in their amino acid sequence and optionally also in their stereochemistry. Thus, for example, a mixture of the invention may comprise a plurality or two or more recognition peptides, each comprising 5 amino acids (a 5-mer peptide), being leucine and lysine, wherein the two or more peptides differ in the sequence of leucine and lysine. Where a recognition peptide is conjugated to a fatty moiety, the mixture may contain conjugated and non-conjugated peptides.

The recognition surfaces of the invention may be used as sensors or implemented in sensor devices for detecting presence, and/or amount and/or viability of a microorganism in a liquid sample, an aerosol or a gas phase. Thus, the invention further provides a sensor comprising a plurality of surface-associated recognition peptides, which may be same or different (RPM), where each of the recognition peptide molecules is capable of interacting or associating with a microorganism.

The sensor of the invention may be formed on a surface region of any solid substrate, which may be a flat substrate, a flexible substrate, a transparent substrate, a 3D substrate, a conductive substrate, a non-conductive substrate or any other solid substrate as known in the art. Depending on the substrate composition, association to the peptides may be through a surface region of the substrate which may be formed of a material that is same or different from that of the substrate. In other words, the substrate may have a surface region that is made of a different material. Typically, the substrate may be formed of any material, and have a surface material that is formed of a material selected from oxides, glass, metal, carbon allotropes and glassy carbon.

In some embodiments, the surface and substrate materials are the same. In some embodiments, the surface is a gold surface.

In some embodiments, the surface is a gold surface or a surface of a glassy carbon electrode (GCE) and the recognition peptide is substituted by a sulfur containing group, e.g., a thiol or a disulfide group.

In some embodiments, the surface is a gold surface or GCE and the recognition peptide is substituted by a carboxy group.

The invention further provides an electrode or electrode array, wherein the electrode or at least one electrode in an electrode array comprises a surface region that is a recognition surface according to the invention.

In some embodiments, the electrode is a gold or a GCE electrode or an electrode useful in electrochemical impedance spectroscopy (EIS), whereby a surface region of the electrode is associated with a monolayer of one or more recognition peptides as disclosed herein. In some embodiments, the recognition peptide is one of TSTWNLYVPPEP (SEQ ID NO 2), WPGPARTQYHRY (SEQ ID NO 3), WPGPARTQYHAY (SEQ ID NO 4), RMYQSLLAPS (SEQ ID NO 5), YSSATFRLHNIS (SEQ ID NO 6), and SYALSSASVRTK (SEQ ID NO 7). In some embodiments, the recognition peptide is TSTWNLYVPPEP or WPGPARTQYHRY (SEQ ID NO 3) or WPGPARTQYHAY (SEQ ID NO 4). In some embodiments, the recognition peptide is one of TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4) that is C- or N-associated to -Gly-Gly-Gly-Cys (-GGGC).

In some embodiments, the electrode is a gold or a GCE electrode or an electrode useful in electrochemical impedance spectroscopy (EIS), whereby a surface region of the electrode is associated with a monolayer of one or more recognition peptides selected from:

(SEQ ID NO 8)
TSTWNLYVPPEP-Gly-Gly-Gly-Cys,
(SEQ ID NO 9)
RMYQSLLAPS-Gly-Gly-Gly-Cys,
(SEQ ID NO 10)
YSSATFRLHNIS-Gly-Gly-Gly-Cys,
(SEQ ID NO 11)
SYALSSASVRTK-Gly-Gly-Gly-Cys,
(SEQ ID NO 12)
WPGPARTQYHRY-Gly-Gly-Gly-Cys,
(SEQ ID NO 13)
WPGPARTQYHAY-Gly-Gly-Gly-Cys,
(SEQ ID NO 14)
Cys-Gly-Gly-Gly-TSTWNLYVPPEP,
(SEQ ID NO 15)
Cys-Gly-Gly-Gly-RMYQSLLAPS,
(SEQ ID NO 16)
Cys-Gly-Gly-Gly-YSSATFRLHNIS,
(SEQ ID NO 17)
Cys-Gly-Gly-Gly-SYALSSASVRTK,
(SEQ ID NO 18)
Cys-Gly-Gly-Gly-WPGPARTQYHRY,
and
(SEQ ID NO 19)
Cys-Gly-Gly-Gly-WPGPARTQYHAY,

wherein in each of the aforementioned peptides, the Cys group is optionally a surface binding group.

In some embodiments, surface of conducting oxides or II-IV and II-VI semiconductor materials may alternatively be used. For such surfaces surface binding groups such as chloro- and alkoxy-silanes, phosphates, catechol groups, and others may be used.

In some embodiments, the electrode is an array of electrodes comprising electrodes of the same type and/or electrodes modified in the same way, e.g., modified with the same recognition peptide. In some embodiments, the electrode is an array or an assembly of different types of electrodes, each type of electrode modified with a different specific peptide for multiplex detection. In some embodiments, an array of electrodes is provided which comprises two or more different types of recognition peptides (RPM).

In some embodiments, an array of electrodes comprises gold-based electrodes. In some embodiments, the array comprises a GCE-based electrode. In some embodiments, the array comprises a gold-based electrode and a GCE-based electrode.

The invention further provides a detection device, e.g., an electrochemical impedance detection device, comprising an electrode according to the invention. The device may be in a form of a biosensor device which employs an electrode having a monolayer of a recognition peptide or RPM in combination with impedance measuring elements integrated into the device. The recognition peptide may be incorporated onto the surface of the electrode and a liquid sample may then be flown or brought into contact with the surface of the electrode. A change in the detected impedance generally indicates microorganism binding or interaction to said recognition peptide.

The detection device of the invention, being in some configurations a device for measuring a change in an electrochemical impedance of a surface, is structured with an electrode of the invention to enable detection and analysis of biorecognition events that occur due to interactions of the recognition peptides with a microorganism, e.g., which presence and/or concentration and/or viability is to be determined. The interaction may be used to derive information as to the occurrence of the interaction, the type of interaction, the agent interacting with the surface, the degree and rate of interaction, the concentration of the agent present, presence of different agents, changes in the interactions over time and others. The device of the invention may be configured to detect interactions occurring online in, e.g., water reservoirs, as in a continuous fashion in facilities or environment having a tendency or a risk of being contaminated, e.g., during industrial processes. Devices of the invention may be provided as bench-top detection devices or as lab-on-a-chip devices.

Thus, a device of the invention may be exploited in a variety of fields and forms, in a continuous detection or pulsed detection protocols, for achieving an effective quantitative and qualitative detection of microorganisms, e.g., viruses, bacteria and fungi.

A difference in an electrical signal as compared to a base signal measured prior to a potential interaction between a microorganism and an electrode surface may occur due to kinetic binding of the microorganism to the peptide monolayer or due to any change in the composition of the monolayer. As a result, electron transfer/charge transfer resistance is produced, representing the amount or concentration of bound microorganism.

Where a device of the invention comprises two or more electrodes or an array of such electrodes, both high sensitivity and high selectivity may be achievable. Various types of recognition peptides can be used to differentiate between different microorganisms. Also, devices and methods of the invention may be used to distinguish, quantify or generally evaluate the viability of a microorganism population or a change in the population viability over time. As dead microorganisms, or non-viable microorganisms, do not induce an electrochemical change (a change in an EIS signal), the viability or a change (increase or decrease) in the viability of a microorganism population may be determined, as disclosed herein.

The “microorganism” which presence, amount and/or viability is to be determined or evaluated may be any noncellular or unicellular (including colonial) organism. The microorganisms may include all prokaryotes. Examples of microorganisms include bacteria (including cyanobacteria), gram-negative organisms, gram-positive organisms, yeasts, spores, lichens, fungi, protozoa, virinos, viroids, viruses, phages, and some algae.

In some embodiments, the microorganism is selected from a bacterium, a fungus, and a virus. In some embodiments, the microorganism is a bacterium or a virus.

Non-limiting examples of microorganisms include:
bacteria from the genus Aeromonas (e.g. A. hydrophilia), Arcobacter, Bacillus (e.g. B. cereus), Brochothrix (e.g. B. thermosphacta), Campylobacter (e.g. C. jejuni), Camobacterium (e.g. C. piscicola), Clostridium (e.g. C. perfringens, C botulinum), Enterobacteriacae, Escherichia (e.g. E. coli), Listeria (e.g. L. monocytogenes), Pseudomonas (e.g. P. putida, P. fluorescens), Salmonella (e.g. S. Typhimurium), Serratia (e.g. S. liquefaciens), Shigella, Staphylococcus (e.g. S. aureus), Vibrio (e.g. V. parahaemolyticus, V. cholerae) and Yersinia (e.g. Y. enterocolitica); Erwinia, Pseudomonas pyocyanea, and Corynebacterium xerosis,
fungi such as Aspergillus flavum and Penicillium chrysogenum; parasites such as Entamoeba (Entamoeba histolytica), Balantidium (Balantidium cob), Cryptosporidium (e.g., Cryptosporidium parvum), Cyclospora (e.g., Cyclospora cayetanensis), Giardia (e.g. Giardia lamblia, Giardia intestinalis), Isospora (Isospora belle), Microsporidia (Enterocytozoon bieneusi, Septata intestinalis), Trichinella spiralis and Toxoplasma gondii; Fusarium oxysporum, Penicillium italicum, Colletotrichum gloeosporioides, Colletotrichum capsica, and Fusarium solani, Pythium, Pythium sp., Sclerotium rolfsii.
viruses and infective viron of viruses selected from bacteriophages, coronaviridae/corona-virus, orthomyxoviridae, paramyxoviridae, Coxsackie family of viruses and adenoviridae family; including corona-virus, such as a COVID-19 causing pathogen, e.g., SARS-COV-2, encompassing SARS-COV-2 having mutations that may be found in the entire genome of SARS-COV-2 strains, e.g., in the 5′ UTR, ORFlab polyprotein, intergenic region, envelope protein, matrix protein and nucleocapsid protein, Tobamovirus, Tomato brown rugose fruit virus.

In some embodiments, the microorganisms is a Gram positive bacterium or a Gram negative bacteria.

In some embodiments, the microorganisms is a Gram positive bacterium such as staphylococci, streptococci and Listeria species.

In some embodiments, the bacterium is methicillin-resistant Staphylococcus aureus (MRSA) or is E. Coli.

In some embodiments, the microorganisms is a Gram negative bacterium such as Enterobacter species, Salmonella species and Pseudomonas species.

In some embodiments, the bacterium is Pseudomonas aeruginosa, e.g., PAO1 or Methicillin resistant S. aureus MRSA, Listeria monocytogenes, Mycoplasma.

Devices and methods of the invention may implement any monolayer of recognition peptides, including recognition peptides comprising or consisting such peptides having sequences here in identified SEQ ID NOS 1, 2, 3, 4, 5, 6, 7, 8, 9, 10, 11, 12, 13, 14, 15, 16, 17, 18, 19, each of which constituting an independent embodiment of the invention. each of these peptides may be provided in combination with other peptides.

Devices and methods of the invention are tailored for determining at least one microorganism-associated parameter of a microorganism present in a sample. The sample may be a liquid medium, including stationary, flowing, sampled or air borne liquid, e.g., droplets of an aerosol, or a gas phase sample that contains microorganisms or is suspected of containing a microorganism. Such a sample may contain the microorganism or may be a sample formed by sampling a solid surface or solid medium or a gas, by flowing the sample into a liquid medium, e.g., by bubbling, or by contacting an object with a liquid that is thereafter tested. Thus, a sampling medium may be a liquid medium that has been brought into contact with a solid, liquid or gaseous environment or body suspected or known to contain a microorganism.

The sample may be a body fluid such as a blood, plasma, urine, salvia, cerebrospinal fluid, sperm, milk sample and others, which may be tested for clinical purposes; a beverage such as a juice, milk, wine, bear and others, with regard to which a determination of contamination may be desired; any liquid sample, such as water, waste water, industrial spills and others, that may come in contact or may be contaminated with a microorganism, for the purpose of determining an environmental microorganism load; liquids used in manufacturing of sensitive products such as food, drug products and cosmetic products; liquid media obtained in a form of samples or swabs from surfaces tested to evaluate a degree of contamination; and others.

In some embodiments, the liquid medium is water or containing water, wherein microorganisms' evaluation is desired for the purpose of determining contamination. Presence, amount or viability may determine, independently or in combination, a degree of contamination by a microorganism. As used herein, devices and methods of the invention are configured to determine at least one “microorganism associated parameter” which may be one or more of presence of the microorganisms in the sample or medium, load or amount or concentration of the microorganism in the sample or medium and viability of the microorganism in the sample or medium, e.g., to determine the efficiency of various sterilization methods. Depending on the particular parameter, the establishment of a conclusive result may be “positive/negative” (or “yes/no”) in case of determining the presence of the microorganism or may be numerical or quantified for microorganism amount and/or viability. As practiced in the art, results and readings received from devices and methods of the invention may be compared to a previously obtained data set or a pre-established calibration curve on the basis of which the parameter may be established or determined.

While devices and methods of the invention are configured to determine at least one microorganism associated parameters, the devices and methods are not necessarily specific for identifying a particular microorganism. Thus, devices and methods may be unselective and determine presence, amount and/or viability of any type of a microorganism.

In some embodiments, devices and methods implementing RPM in a recognition surface of the invention may be used in a method of non-selectively determining presence, amount and/or viability of at least one microorganism in a sample.

In some embodiments, devices and methods implementing RPM in a recognition surface of the invention may be used in a method of non-selectively determining viability or a change in the viability (increase or decrease) of at least one microorganism in a sample.

In some embodiments, devices and methods implementing a peptide consisting or comprising a sequence TSTWNLYVPPEP (SEQ ID NO 2), or RMYQSLLAPS (SEQ ID NO 5), or YSSATFRLHNIS (SEQ ID NO 6), or SYALSSASVRTK (SEQ ID NO 7), or WPGPARTQYHRY (SEQ ID NO 3), or WPGPARTQYHAY (SEQ ID NO 4) may be used for determining presence, amount and/or viability of a microorganism selected from bacteria. In some embodiments, the bacteria is Gram negative or a Gram positive bacteria. In some embodiments, the bacterium is selected from MRSA, E. coli (such as E. coli RP) or PAO1.

In some embodiments, the peptide is TSTWNLYVPPEP (SEQ ID NO 2), or WPGPARTQYHRY (SEQ ID NO 3), or WPGPARTQYHAY (SEQ ID NO 4). In some embodiments, the recognition peptide is one of TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4) that is C- or N-associated to -Gly-Gly-Gly-Cys, as disclosed herein.

Recognition surfaces and thus sensors or devices of the invention may be fabricated by depositing a population of recognition peptides having each a surface binding group onto a surface region of a substrate and allowing said recognition peptides to form a monolayer on said surface region. As noted herein, the surface binding group is selected based on the surface region onto which the association is to be formed, or the substrate is selected based on the surface binding groups that are used.

The monolayer formation may involve molecular self-assembly, namely spontaneous formation of binding interactions between the surface binding groups of the recognition peptides and the surface material. Thus, the invention further provides a method for fabricating a recognition surface or a sensor device according to the invention, the method comprising forming by self-assembly on a surface region of a substrate a monolayer comprising a plurality of recognition peptides, wherein the assembly of the recognition peptides onto the surface region is achievable via a surface binding group present on the recognition peptide.

As noted herein, the peptide may be a single type of peptide or a mixture of peptides such as an RPM. In some embodiments, the recognition peptide is any one peptide selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4). In some embodiments, the recognition peptide comprises a surface binding group. In some embodiments, the recognition peptide is associated to a surface binding group via a linker group, as disclosed herein. In some embodiments, the surface binding group is a cysteine group. In some embodiments, the recognition peptide is one of TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4) that is C- or N-associated to -Gly-Gly-Gly-Cys. In some embodiments, the recognition peptide is any of the specifically indicated peptides disclosed herein.

In some embodiments, the method of fabricating a recognition surface comprising contacting a substrate material with a solution comprising a single population or a mixture of recognition peptides, and permitting self-assembly of said peptides onto a surface region of said substrate.

In some embodiments, the method comprises obtaining a population of recognition peptides as disclosed herein.

The self-assembly is typically spontaneous and may involve covalent, electrostatic, π-binding, or may be achieved by electrochemical grafting. Methods of self-assembly may vary, all of which may be applicable to constructing a monolayer of the invention. Such methods are known in the art, for example in review papers such as Self-Assembly Peptides-a Review, Ayanda et al., J. of Biol. Sci, 2022, pp 309-322; and textbooks such as Peptide Self-Assembly, Methods and Protocols, Nilsson et al., 2018.

In some embodiments, the substrate is an electrode as defined herein. In some cases, the electrode is configured for use in a variety of methods of use.

In another aspect, the invention provides a method for determining presence of a microorganism in a liquid sample, the method comprising contacting an electrode having at least a surface region thereof associated with a monolayer of a recognition peptide, as disclosed herein, with a liquid sample containing or suspected of containing said microorganism and determining a change in an impedance signal generated from a base signal obtained for a control sample. The change generated may be indicative of the presence of the microorganism.

Without limitations, the control sample used for determining a set point, a threshold or a background signal from which a signal may be attributed to the microorganism may be determined, may be a carrier medium identical to that of the sample known not to contain the microorganism, an identical sample known not to contain the microorganism, or generally any microorganism-free sample. For example, where presence of microorganism in a biological sample, e.g., blood sample, is desired, a blood sample known not to contain the microorganism may be used as a control. Similarly, for determining concentration of a microorganism or an improvement in a medical treatment following or during treatment, a subject's blood samples may be tested on numerous occasions during a period the subject is undergoing the medical treatment to detect changes in the concentration of the microorganism, as determined by a change in impedance, whereby such a change may indicate a reduction in the amount of microorganism present in the blood of the subject or in the viability of the microorganism. A determination of concentration may be based on predetermined studies using varying known concentrations of the microorganism to be detected, as known and practiced in the art. The change in the impedance signal as compared to a base signal generated for a control sample provides an indication of an interaction between a microorganism and the recognition or electrode surface. The change on the signal may be an increase or a decrease in the impedance signal relative to a base line of the recognition surface under predetermined conditions.

The invention further provides a method for determining an amount or a concentration of a microorganism in a sample, the microorganism being capable of interacting with a recognition surface or an electrode comprising a population of recognition peptides, the method comprising contacting said surface or electrode with the sample, measuring a change in an impedance signal relative to an impedance signal measured for one or more control samples having known concentrations of the microorganism and determining concentration of the microorganism in the sample.

Methods of the invention may be used for clinical purposes, in the food and agricultural industries as well as in the cosmetic industry. In a medicinal setting (to determine sterility of instrument and surfaces) in the context of environmental and agricultural setting and as a pre step for determining eligibility of a subject effected with microorganism infection to treatment with a recognition peptide.

The invention further provides a method for determining a level or concentration of viable microorganisms in a sample, the method comprising contacting a surface or an electrode of the invention with the sample, measuring a change in an impedance signal relative to an impedance signal measured for one or more control samples having known concentrations of the microorganism and determining a change in the level or concentration of the microorganism in the sample, wherein a reduction in the impedance signal indicates a reduction or an increase the viable microorganism population in the sample.

In some embodiments, the method is used for determining viability of bacteria in a sample for determining efficiency of a sterilization protocol, e.g., used in a manufacturing process (pasteurization) or in medicinal setting (autoclave). In such a case the presence of live microorganisms beyond a pre-defined level provides an indication that the sterilization was not successful.

Based on the disclosure herein, aspects and embodiment of the invention include, inter alia:

An electrochemical impedimetric recognition surface, comprising a substrate having a surface associated with a monolayer of recognition peptides capable of interacting with a microorganism present in a sample, wherein said interaction between the layer of recognition peptides and the microorganism induces a surface change detectable by electrochemical impedance spectroscopy (EIS).

A recognition surface is a surface of an electrode, a sensor or an electrochemical device.

A recognition surface, wherein the monolayer is of a single type of recognition peptide or a mixture of recognition peptides.

A recognition surface, wherein each of the recognition peptides having a surface binding group associating each of the recognition peptides to the surface.

A recognition surface, wherein the surface binding group is selected from amines, carboxylic acids, thiols, disulfides, and phosphates.

A recognition surface, wherein each of the recognition peptides having a sulfur-containing group associating said recognition peptides to the surface.

A recognition surface, wherein the sulfur-containing group is a thiol or a disulfide.

A recognition surface, wherein the sulfur containing group is an amino acid containing a sulfur atom.

A recognition surface, wherein the surface is a gold surface or a surface of a glassy carbon electrode (GCE).

A recognition surface, wherein each of the recognition peptides comprises a linker moiety of 1 to 5 amino acids.

A recognition surface, wherein the linker moiety is or comprises glycine (Gly) and/or cysteine (Cys).

A recognition surface, wherein each of the recognition peptides comprises glycine and/or cysteine.

A recognition surface, wherein at least a portion or all of the recognition peptides comprises a linker moiety in a form of a tetrapeptide.

A recognition surface, wherein the tetrapeptide is -Gly-Gly-Gly-Cys (-GGGC), wherein Cys (C) is a surface binding group.

A recognition surface, wherein the monolayer comprises or consists a plurality of recognition peptides, each comprising between 8 and 50 amino acids.

A recognition surface, wherein the monolayer comprises or consists a plurality of recognition peptides, each comprising an amino acid sequence selected from

(SEQ ID NO 2)
TSTWNLYVPPEP,
(SEQ ID NO 3)
WPGPARTQYHRY,
(SEQ ID NO 4)
WPGPARTQYHAY,
(SEQ ID NO 5)
RMYQSLLAPS,
(SEQ ID NO 6)
YSSATFRLHNIS,
and
(SEQ ID NO 7)
SYALSSASVRTK

A recognition surface, wherein the monolayer comprises or consists a plurality of recognition peptides, each comprising an amino acid sequence being TSTWNLYVPPEP (SEQ ID NO 2), or WPGPARTQYHRY (SEQ ID NO 3), or WPGPARTQYHAY (SEQ ID NO 4), or RMYQSLLAPS (SEQ ID NO 5), or YSSATFRLHNIS (SEQ ID NO 6), or SYALSSASVRTK (SEQ ID NO 7).

A recognition surface, wherein the recognition peptide comprises the sequence

(SEQ ID NO 2)
TSTWNLYVPPEP,
or
(SEQ ID NO 3)
WPGPARTQYHRY,
or
(SEQ ID NO 4)
WPGPARTQYHAY

A recognition surface, wherein the recognition peptide comprises a linker moiety consisting or comprising between 1 and 5 amino acids, wherein said liker moiety is covalently associated with an amino acid sequence selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4).

A recognition surface, wherein the monolayer comprises a plurality of recognition peptides, each comprises an amino acid selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4) that is associated to a tetrapeptide of the form-Gly-Gly-Gly-Cys (-GGGC), wherein Cys (C) is a surface binding group.

A recognition surface, wherein the monolayer comprising or consisting a plurality of recognition peptides having sequence TSTWNLYVPPEP-Gly-Gly-Gly-Cys (SEQ ID NO 8).

A recognition surface, wherein the monolayer comprising or consisting a plurality of recognition peptides having sequence RMYQSLLAPS-Gly-Gly-Gly-Cys (SEQ ID NO 9).

A recognition surface, wherein the monolayer comprising or consisting a plurality of recognition peptides having sequence YSSATFRLHNIS-Gly-Gly-Gly-Cys (SEQ ID NO 10).

A recognition surface, wherein the monolayer comprising or consisting a plurality of recognition peptides having sequence SYALSSASVRTK-Gly-Gly-Gly-Cys (SEQ ID NO 11).

A recognition surface, wherein the monolayer comprising or consisting a plurality of recognition peptides having sequence WPGPARTQYHRY-Gly-Gly-Gly-Cys (SEQ ID NO 12).

A recognition surface, wherein the monolayer comprising or consisting a plurality of recognition peptides having sequence WPGPARTQYHAY-Gly-Gly-Gly-Cys (SEQ ID NO 13).

A recognition surface, wherein the monolayer comprising or consisting a plurality of recognition peptides having sequence Cys-Gly-Gly-Gly-TSTWNLYVPPEP (SEQ ID NO 14).

A recognition surface, wherein the monolayer comprising or consisting a plurality of recognition peptides having sequence Cys-Gly-Gly-Gly-RMYQSLLAPS (SEQ ID NO 15).

A recognition surface, wherein the monolayer comprising or consisting a plurality of recognition peptides having sequence Cys-Gly-Gly-Gly-YSSATFRLHNIS (SEQ ID NO 16).

A recognition surface, wherein the monolayer comprising or consisting a plurality of recognition peptides having sequence Cys-Gly-Gly-Gly-SYALSSASVRTK (SEQ ID NO 17).

A recognition surface, wherein the monolayer comprising or consisting a plurality of recognition peptides having sequence Cys-Gly-Gly-Gly-WPGPARTQYHRY (SEQ ID NO 18).

A recognition surface, wherein the monolayer comprising or consisting a plurality of recognition peptides having sequence Cys-Gly-Gly-Gly-WPGPARTQYHAY (SEQ ID NO 19).

A recognition surface, wherein the monolayer comprising or consisting a plurality of recognition peptides being a random peptide mixture, RPM.

A recognition surface, wherein the RPM comprises a mixture of two types of peptides:
recognition peptides having between 5 and 20 amino acids selected from hydrophobic amino acid and a cationic amino acid; and
conjugates of the recognition peptides covalently associated to fatty moieties or glycans.

A recognition surface, for use in manufacturing an electrode or a sensing device.

An electrode for use in electrochemical impedance spectroscopy (EIS), the electrode having a recognition surface according to the invention.

An electrochemical impedance spectroscopy (EIS) electrode, the electrode having a recognition surface according to the invention.

An electrochemical impedance spectroscopy (EIS) electrode, the electrode having a gold surface associated with a monolayer of recognition peptide molecules of the form PP-Gly-Gly-Gly-Cys, wherein the association is through the Cys group and wherein PP is a recognition peptide capable of interacting with a microorganism in a liquid medium.

An electrode, wherein the recognition peptide is a random peptide mixture (RPM) or a peptide selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4).

A sensor comprising a substrate having a surface-associated with a plurality of recognition peptides, wherein the recognition peptides selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4).

A sensor, wherein the substrate having a gold surface.

A sensor, wherein the substrate is a glassy carbon electrode (GCE).

A detection device comprising an electrode according to the invention.

A device being an EIS device.

A device for measuring a change in an electrochemical impedance of a surface comprising surface-associated recognition peptides interacting with a microorganism in a sample.

A device for determining presence and/or concentration and/or viability of a microorganism in a sample.

A device, wherein the sample is a liquid sample or a gaseous sample.

A device, wherein the microorganism is a noncellular or unicellular organism.

A device, wherein the microorganism is bacteria, gram-negative organisms, gram-positive organisms, yeasts, spores, lichens, fungi, protozoa, virinos, viroids, viruses, phages, and algae.

A device, wherein the microorganism is selected from a bacterium, a fungus, and a virus.

A device, wherein the microorganism is a bacterium or a virus.

A device, wherein the microorganism is Gram positive bacteria or Gram negative bacteria.

A device, wherein the microorganism is Gram positive bacteria selected from staphylococci, streptococci and Listeria species.

A device, wherein the microorganism is a methicillin-resistant Staphylococcus aureus (MRSA) or is E. Coli.

A device, wherein the microorganism is Gram negative bacteria selected from Enterobacter species, Salmonella species and Pseudomonas species.

A device, wherein the microorganism is Pseudomonas aeruginosa, PAO1, Methicillin resistant S. aureus MRSA, Listeria monocytogenes, or Mycoplasma.

A method for determining at least one microorganism-associated parameter of a microorganism present in a sample, the method comprising contacting an electrochemical impedimetric recognition surface or a device implementing same with a sample containing or suspected of containing the microorganism, wherein the recognition surface comprises a substrate having a surface associated with a layer of recognition peptides capable of interacting with the microorganism present or suspected of being contained in the sample, wherein said interaction between the layer of recognition peptides and the microorganism induces a surface change indicative of said interaction.

A method, wherein the sample is a liquid sample, an aerosol or a gaseous sample.

A method, wherein said liquid sample is obtained by collecting a sample from a solid, a liquid or a gaseous medium into a liquid carrier, being optionally water.

A method, wherein the method is carried in on a stationary or a flowing liquid sample.

A method, wherein the liquid sample is a body fluid sample obtained from a subject.

A method, wherein the body fluid sample is blood, plasma, urine, salvia, cerebrospinal fluid, sperm, and human or animal milk.

A method, wherein the liquid medium is a beverage or a food product.

A method, wherein the liquid medium is liquid used in manufacturing of sensitive products prone to microbial contamination.

A method, wherein the sensitive product is food, drug products and cosmetic products.

A method, for determining microbial contamination in an industrial facility or an environment.

A method, wherein the determination is timed or continuous.

A method, wherein the microorganism associated parameter is one or more of presence of the microorganisms in the sample, load or amount or concentration of the microorganism in the sample, and viability of the microorganism in the sample.

A method, wherein the recognition peptide is selected from TSTWNLYVPPEP (SEQ ID NO 2), RMYQSLLAPS (SEQ ID NO 5), YSSATFRLHNIS (SEQ ID NO 6), SYALSSASVRTK (SEQ ID NO 7), WPGPARTQYHRY (SEQ ID NO 3), and WPGPARTQYHAY (SEQ ID NO 4).

A method, wherein the recognition peptide is associated to a surface binding linker having the structure Gly-Gly-Gly-Cys.

A method for determining presence, amount and/or viability of a bacterium.

A method, wherein the bacterium is selected from Gram negative and Gram positive bacteria.

A method, wherein the bacterium is selected from MRSA, E. coli, E. coli RP, and PAO1.

A method, wherein the recognition peptide is a random peptide mixture (RPM).

A method for determining viability of a microorganism in the sample.

A method for non-selectively determining viability of a microorganism in the sample.

A method for determining a change (increase or decrease) in the viability of the microorganism in a sample.

A method for determining a reduction in microorganism population following medical treatment.

A method for determining a reduction in microorganism population or microorganism responsivity to a medical treatment in vivo or in vitro.

A method for determining an amount or a concentration of a microorganism in a sample, the method comprising contacting said surface with the sample, measuring a change in an impedance signal relative to an impedance signal measured for one or more control samples having known concentrations of the microorganism and determining concentration of the microorganism in the sample.

A method for determining a level or concentration of viable microorganisms in a sample, the method comprising contacting the recognition surface with the sample, measuring a change in an impedance signal relative to an impedance signal measured for one or more control samples having known concentrations of the microorganism and determining a change in the level or concentration of the microorganism in the sample, wherein a reduction in the impedance signal indicates a reduction or an increase in the viable microorganism population in the sample.

A method for determining viability of bacteria in a sample for determining efficiency of a sterilization protocol.

A method, wherein the microorganism is a noncellular or unicellular organism.

A method, wherein the microorganism is bacteria, gram-negative organisms, gram-positive organisms, yeasts, spores, lichens, fungi, protozoa, virinos, viroids, viruses, phages, and algae.

A method, wherein the microorganism is selected from a bacterium, a fungus, and a virus.

A method, wherein the microorganism is a bacterium or a virus.

A method, wherein the microorganism is Gram positive bacteria or Gram negative bacteria.

A method, wherein the microorganism is Gram positive bacteria selected from staphylococci, streptococci and Listeria species.

A method, wherein the microorganism is a methicillin-resistant Staphylococcus aureus (MRSA) or is E. Coli.

A method, wherein the microorganism is Gram negative bacteria selected from Enterobacter species, Salmonella species and Pseudomonas species.

A method, wherein the microorganism is Pseudomonas aeruginosa, PAO1, Methicillin resistant S. aureus MRSA, Listeria monocytogenes, or Mycoplasma.

The invention further contemplates recognition peptides selected from:
PP-Gly-Gly-Gly-Cys (PP-GGGC, SEQ ID NO 1), wherein PP is any recognition peptide comprising a plurality of amino acids, as defined herein,

(SEQ ID NO 2)
TSTWNLYVPPEP,
(SEQ ID NO 3)
WPGPARTQYHRY,
(SEQ ID NO 4)
WPGPARTQYHAY,
(SEQ ID NO 5)
RMYQSLLAPS,
(SEQ ID NO 6)
YSSATFRLHNIS,
(SEQ ID NO 7)
SYALSSASVRTK,
(SEQ ID NO 8)
TSTWNLYVPPEPGGGC,
(SEQ ID NO 9)
RMYQSLLAPSGGGC,
(SEQ ID NO 10)
YSSATFRLHNISGGGC,
(SEQ ID NO 11)
SYALSSASVRTKGGGC,
(SEQ ID NO 12)
WPGPARTQYHRYGGGC,
(SEQ ID NO 13)
WPGPARTQYHAYGGGC,
(SEQ ID NO 14)
CGGGTSTWNLYVPPEP,
(SEQ ID NO 15)
CGGGRMYQSLLAPS,
(SEQ ID NO 16)
CGGGYSSATFRLHNIS,
(SEQ ID NO 17)
CGGGSYALSSASVRTK,
(SEQ ID NO 18)
CGGGWPGPARTQYHRY,
and
(SEQ ID NO 19)
CGGGWPGPARTQYHAY.

Each of the above recognition peptides constitutes an independent aspect of the invention.

The invention further contemplates use of any of the above recognition peptides in any aspect, method, process of use, recognition surface, electrode, device or system according to the invention. For example, the invention contemplates use of any of the recognition peptides in an impedimetric method of analysis, or in a method of determining presence, amount and/or viability of a microorganism in a sample, or for constructing an electrode for EIS.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Solid phase peptide synthesis of phenylalanine-lysine random peptide mixtures with cysteine at the C terminus (first coupling step, yellow circle). After the first coupling of cysteine, 1:1 mixture of Fmoc-phenylalanine and Fmoc-lysine was coupled at each of the 20 coupling steps to generate 21-mer CFK.

FIGS. 2A-B: AFM analysis of the modified gold substrates. (A) Bare gold (Ra=0.26 nm); (B) after adsorption CFK random peptide mixtures (Ra=0.31 nm).

FIG. 3: MALDI TOF/TOF analysis of CFK. Averaged expected molecular mass is 2875.639 Da. The main peak corresponds to 21-mer with distribution of F:K ratio.

FIG. 4: Reductive desorption of FKC modified Au electrode. The CV recorded in 0.1 M KOH solution from −0.5 to 1.4 V at the scan rate of 150 mV

Au−SR+e−→Au+SR⁻

From the CV data, we were able to calculate the density of FK molecules according to equation:

$$F K = \frac{Q * C}{A}$$

FK is the amount of FK molecules, Q is the charge transfer (calculated by the integral of the CV peak), C is the coulomb constant and A is the area of the electrode. We obtain FKC surface concertation of 2.5 molecules/nm²=1.4*10⁻¹⁰ moles/cm².

FIGS. 5A-B: AFM analysis of the modified gold substrates. (A) Bare gold (Ra=0.26 nm); (B) after adsorption CFK random peptide mixtures (Ra=0.31 nm).

FIGS. 6A-C: XPS spectra of CFK modified Au surface: A) C 1s BE region, B) N 1s BE region, C) S 2p BE region (top lines) and bare Au surface (bottom lines). The measured data is shown in lines and the dash lines represent the Gaussian fit.

FIG. 7: Binding of various bacteria to Au-CFK. Bacteria stained with syto9, incubated on CFK modified surface, the unbound bacteria were washed and the surfaces were observed in fluorescence microscope, X20 magnification.

FIG. 8: Coverage of FKC surfaces with 10⁸ and 10⁶ CFU/mL MRSA and E. coli in 2 magnifications as observed by EVOS fluorescence microscopy. Scale bar: magnification ×40—75 μM, magnification ×10—300 μM.

FIG. 9: Binding of bacteria to bare Au as observed by fluorescence microscopy, magnification X20.

FIGS. 10A-B: Impedimetric response of AuE-CFK to exposure to bacteria. A) Nyquist plot of AuE-CFK before (Black) and after (Red) a 40 min incubation with E. coli. B) Normalized R_{CT} values of AuE-CFK after exposure to bacteria. Results are Avg+SD of 3 different electrodes with 3 exposure repeats.

FIGS. 11A-E: Nyquist plot of AuE-CFK before (Black) and after 40 min (Red) exposure to A. PBS B. PAO1 C. E. coli D. MRSA E. B. subtilis. The change presented as Normalized RCT. Results are Avg+SD of 3 experiments with 3 biological experiments.

FIG. 12: Live dead staining of various bacteria on FKC-Au surface.

FIG. 13: EIS measurements of dead and live PAO1. PAO1 was killed using addition of 100 μg/mL FKC to bacteria suspension.

FIGS. 14A-B: Effect in [Fe(CN)₆]^{3-/4-} on the binding of bacteria to FKC surfaces. A. after 10 min incubation with bacteria. B. After additional incubation with [Fe(CN)₆]^{3-/4-}. Magnification ×10, scale bar −300 μM.

FIG. 15: Peptide having the sequence WPGPARTQYHRY (peptide WPG) (SEQ ID NO 3) was immobilized on a gold surface via a thiol covalent bond (10 μM), the bacterial cells Methicillin-resistant Staphylococcus aureus (MRSA), Pseudomonas aeruginosa (PAO1) and Escherichia coli (E. coli RP) bacterial cells. were stained with DAPI (4′,6-diamidino-2-phenylindole) and observed via Evos florescence microscopy

FIG. 16: WPGPARTQYHRY (SEQ ID NO 3) binding to Methicillin-resistant Staphylococcus aureus (MRSA), Pseudomonas aeruginosa (PAO1) and Escherichia coli (E. coli RP) and Bacillus cereus (Bc) bacterial cells Flow Cytometry analysis. The bacterial cells ~10\hat{ }8 CFU/mL were incubated with 0.5-50 μM FL-labeled peptide. The unbounded peptides were removed and the amount of FL-labeled bacteria was measured

FIGS. 17A-B: A schematic illustration of immobilization of the peptide to the gold surface. The peptides were synthesized with (Gly-Gly-Gly-Cys) linker at the N′ terminus. The thiol of the cysteine amino acid formed a covalent bond with the gold. FIG. 17B: WPGPARTQYHRY (SEQ ID NO 3) sequence Alanine scan peptides. WPG is the original sequence, in each other peptide, one amino acid was changed to Alanine amino acid. The gray marked sequences were synthesized and purified by HPLC.

FIGS. 18A-B: (FIG. 18A) WPG peptide (P5) and four derived alanine scan peptides binding to Methicillin-resistant Staphylococcus aureus (MRSA), Pseudomonas aeruginosa (PAO1) and Escherichia coli (E. coli RP) bacterial cells. A. Immobilized derived peptides on a gold surface via a thiol covalent bond (10 μM), the bacterial cells were stained with DAPI (4′,6-diamidino-2-phenylindole) and observed via Evos florescence microscopy. B. Flow Cytometry analysis. The bacterial cells ~10\hat{ }8 CFU/mL were incubated with 10 μM FL-labeled peptide. The unbounded peptides were removed and the amount of FL-labeled bacteria was measured. (FIG. 18B) WPGPARTQYHRY (SEQ ID NO 3) and four derived alanine scan peptides binding to Methicillin-resistant Staphylococcus aureus (MRSA), Pseudomonas aeruginosa (PAO1) and Escherichia coli (E. coli RP) bacterial cells. Flow Cytometry analysis. The bacterial cells ~10\hat{ }8 CFU/mL were incubated with 10 μM FL-labeled peptide. The unbounded peptides were removed and the amount of FL-labeled bacteria was measured

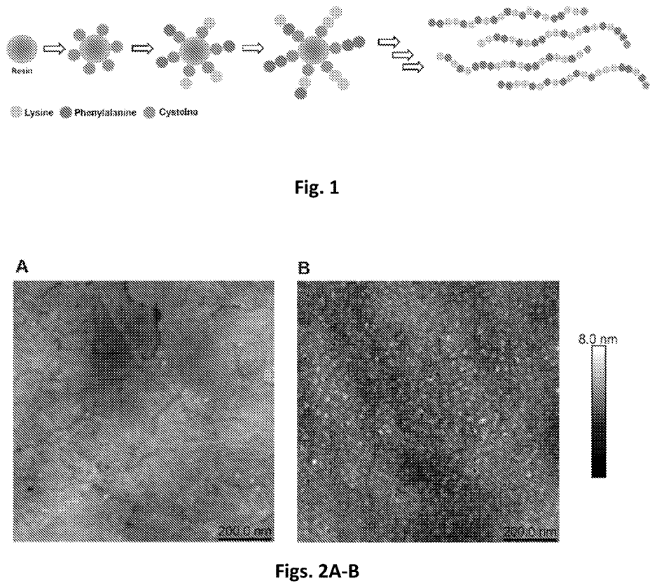

### DETAILED DESCRIPTION OF THE INVENTION

### Experimental

### Materials

All solutions were prepared with triple deionized water (TDW, 18.3 MΩ·cm, Millipore Milli-Q, Bedford, MA). Buffer solution was PBS in low ionic strength (PBS-L) contains 10 mM Na₂HPO₄*7H₂O, 1.8 mM KH₂PO₄, 1.37 mM NaCl and 0.027 mM KCl (pH 7.4). Electrochemical analyses were conducted with Metrohm-Autolab PGSTAT-12 digital potentiostat (EcoChemie BV, Utrecht, The Netherlands) operated with Nova software for utilizing Electrochemical Impedance Spectroscopy (EIS). A three electrodes cell was used for the measurements: Ag/AgCl (in 3 M KCl) as reference electrode (RE) and Pt wire as a counter electrode (CE). Polycrystalline bulk gold electrodes with a 2 mm diameter were used as working electrodes (WE) (CH instruments). WE were manually polished on micro-cloth pads (Buehler, Lake Bluff, IL) with de-agglomerated 0.05 μm alumina suspension (Buehler). After polishing the electrodes were washed with TDW. Bacterial strains used in the research are E. coli K12 MG1655 (rp) and Pseudomonas aeruginosa PAO1, Staphylococcus aureus Newmann (kindly received from Prof. Roni Shapira's lab, HUJI), Methicillin resistant Staphylococcus aureus (MRSA), Bacillus subtilis 168, E. coli K12 BW25113 (kindly received from Prof. Shimshon Belkin's lab, HUJI). All bacteria were grown in luria broth (LB) at 37 °C.

### Methods

### Synthesis of FKC Random Peptide Mixtures (RPMs)

FKC random peptide mixtures were synthesized using standard Fmoc-based solid-phase peptide synthesis (SPPS) with a Liberty Blue™ automated microwave peptide synthesizer (CEM Corp., Matthews, NC, USA) as was published previously (Rreff). (FK)₂₀-Cys was synthetized on 0.25 mmol scale on rink amide resin (loading 0.53 mmol/g (ChemImpex) as the solid support. After swelling (5 min) of the resin, Fmoc removal (deprotection) was done with 20% v/v piperidine/DMF. Standard couplings of amino acids were carried out at 0.1 M in N,N-Dimethylformamide (DMF) using DIC/OxymaPure® activation (0.2 and 0.1 M respectively). Fmoc-Cysteine-Trt was coupled first at 50 °C, followed by deprotection. The next 20 coupling steps were carried out by using 1:1 molar ratio mixture of protected α-amino acids: Fmoc-L-Phenylalanine with Fmoc-L-Lysine-Boc. At end of synthesis, the resin was washed with DMF and DCM and dried with methanol and diethyl ether. The peptides were cleaved from the resin by mixing the resin with 4 mL cleavage cocktail of 92.5% Trifluoroacetic acid (TFA), 2.5% 1,2-Ethanedithiol, 2.5% triisopropylsilane (TIPS), and 2.5% TDW for 3 h with agitation. After filtration, the peptides were precipitated from the TFA solution by the addition of cold diethyl ether and collected by centrifugation (Eppendorf R5810 8000 rpm for 10 min). The ether was then removed, and the process repeated once more. The peptides were dried under a stream of nitrogen, dissolved in 20% ACN/TDW and lyophilized. The product was analyzed by MALDI TOF/TOF AutoFlex Speed (Bruker daltonics) to evaluate the success of synthesis.

### FKC Assembly on Gold Surface

Gold substrates for surface characterization were prepared by evaporation of 10 nm chrome as adhesion layer followed by evaporation of 100 nm Au on top of highly-doped n-type Silicon wafer. The surfaces were washed with ethanol, dried under a mild stream of nitrogen gas and cleaned using UVOCS (ultraviolet ozone cleaning system) for 20 min.

Solution of 1.4 mg/ml (0.5 mM) FKC was prepared in PBS-L buffer solution (pH=7.4). Variety of buffers were examined and finally the adsorption process was done in PBS buffer with low ionic strength (PBS-L) in order to enable the optimal organization and interaction of the peptide's chains. Chemisorption was performed by drop casting FKC solution on bare gold substrates for overnight (16 h) incubation at 37 °C. The surfaces were washed after incubation with PBS-L buffer and TDW and dried under nitrogen stream.

### Surface Characterization

Variable angle spectroscopic ellipsometry (VASE) measurements carried with VB-400 ellipsometer (J.A. Woollam Co., Inc). The thickness of the random peptides layer on gold surface was measured by fitting to CAUCHY model, when the values are an average with standard deviation of surfaces from different batches.

Kelvin probe derived contact potential difference (CPD) measurements were performed with Kelvin probe S vibrating gold grid reference electrode (WF~4.8 eV), operated by Kelvin control 07 unit (DeltaPhi Besocke, Julich, Germany), in a home-built faraday cage under inert argon atmosphere. Ohmic back contacts were made with eutectic Ga—In (99.99%, Sigma-Aldrich) and CPD signal was recorded using Keithley 2450 SMU. The measurements were taken after the few minutes needed for the signal to stabilize, and were performed with respect to reference electrode.

X-ray photoelectron spectroscopy (XPS) spectra were recorded with Kratos Axis Supra⁺ spectrometer (Kratos Analytical Ltd., Manchester, U.K.) using an Al K_{α} monochromatic radiation X-ray source (1486.7 eV). Data were collected and analyzed by using a Casa XPS (Casa Software Ltd.) and Vision data processing program (Kratos Analytical Ltd.). The high-resolution XPS spectra were collected with a take-off angle of 90° (normal to analyzer); vacuum condition in the chamber was 1.9 nTorr, for the C 1s, O 1s, N 1s, S 2p, and Au 4f levels with pass energy of 20 and 0.1 eV step size. The binding energies were calibrated using C 1s peak energy as 285.0 eV.

Reductive desorption analysis conducted by cyclic voltammetry (CV) was performed to determine surface coverage of FK on Au electrodes by scanning over the potential range of −0.5 to −1.4 V at the scan rate of 150 mV/s in 0.1 M KOH solution. Electrolyte solution was degassed with nitrogen steam prior to CV scans.

Atomic force microscopy (AFM) analysis was performed using a Dimension Icon XR probe microscope (Bruker) in tapping mode on ultra-flat gold substrates.

### Florescence Microscopy

Fluorescence microscopy was used to evaluate the binding ability of bacteria to FK modified Au surfaces. Bacteria were grown as described above, then centrifuged and washed with PBS-L twice. The bacteria pellet was diluted to OD₆₀₀ₙₘ=0.5 and stained with Syto9 (live, green) (Life Technologies Corp., Oregon, USA). The suspension was dropped on the peptide immobilized surfaces and incubated for 40 min at RT. The surfaces were washed with PBS-L and TDW (three times) to remove unbound bacteria and observed with fully motorized IX81 fluorescent microscope (Olympus, Japan). Images were captured at magnifications ×20 in at least three distinct areas of the samples. PMT emission 490-530.

### Gold Electrodes Surface Modifications and Impedimetric Sensing

WE were dipped in 0.5 mM (FK)₂₀-Cys in PBS in low ionic strength (PBS-L) contains 10 mM Na₂HPO₄*7H₂O, 1.8 mM KH₂PO₄, 1.37 mM NaCl and 0.027 mM KCl (pH 7.4) for 16 h at 37 °C, then rinsed by dipping in PBS-L and measured. Subsequently, the electrodes were exposed to 10⁸ CFU/mL bacterial cells suspension in PBS-L for 40 min under gentle agitation. The bacteria cells were grown in LB at 37 °C overnight. The bacterial cells suspension was diluted 1/50 in fresh LB and was grown for additional 3-4 h. Bacteria pellets were washed twice with PBS-L by centrifugation (8000 rpm, 2 min) and diluted to optical density of 0.5 at 600 nm. Following incubation with bacteria, the electrodes were washed and measured. The EIS characterization was prepared in EIS solution contained 1 mM K₃[Fe(CN)₆], 1 mM K₄[Fe(CN)₆] (RedOx species) in PBS-L. The spectra were recorded by applying a single sine AC potential of 10 mV amplitude superimposed with 0.21 V DC potential (vs. RE) and scanning over the frequency range of 100 kHz to 0.1 Hz. The data was analyzed as Nyquist plots and fitted to Randle's like equivalent circuit of R_{S}[(R_{CT}W)Q] where R_{S} is the solution resistance, R_{CT} is the interface charge-transfer resistance, W is the Warburg diffusion element, and Q is the constant phase element of non-ideal capacitance. All experiments were done at least on 3 samples with at least 3 repeats.

### Results and Discussion

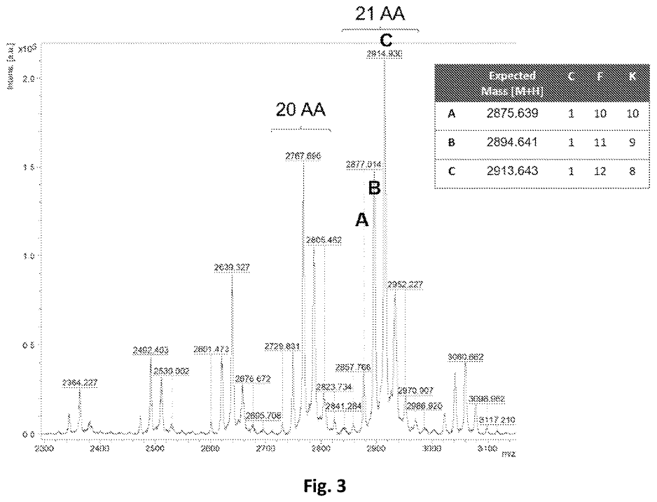

RPMs were synthetized with a 1:1 mixture of phenylalanine and lysine in each coupling step of the solid phase peptide synthesis. The product is a mixture of 2ⁿ possible different sequences composed of phenylalanine and lysine only (FIG. 2). The peptides mixture, termed FK, has antimicrobial activity against broad spectrum of bacteria through binding their membrane. Here, we equipped the FK RPMs with a cysteine moiety at the C terminal to allow anchoring to gold surfaces (CFK). MALDI analysis of CFK presents several peaks around 2.8 kDa, which correlates with a 21-mer peptide composed of 1:10:10 ratio of cysteine, phenylalanine and lysine (FIG. 3).

FK terminated with cysteine at the C-terminal (CFK) was incubated on gold electrodes or surfaces. The thiol chemisorbs onto the gold via formation of S—Au bond and the peptides were self-organized to form a monolayer. The resulting layers were characterized by various surface chemistry analyses.

To characterize the monolayer, CFKs were assembled on Au coated Si wafers (Au-CFK). The variable angle spectroscopic ellipsometry (VASE) analysis showed an additional thickness of 15.3(±0.8) Å (MSE=5.45). The deviation of the measured thickness from the theoretical length of the peptides (~47 Å) indicates that CFK forms a monolayer and that they are either tilted or that the assembly did not reach full coverage.

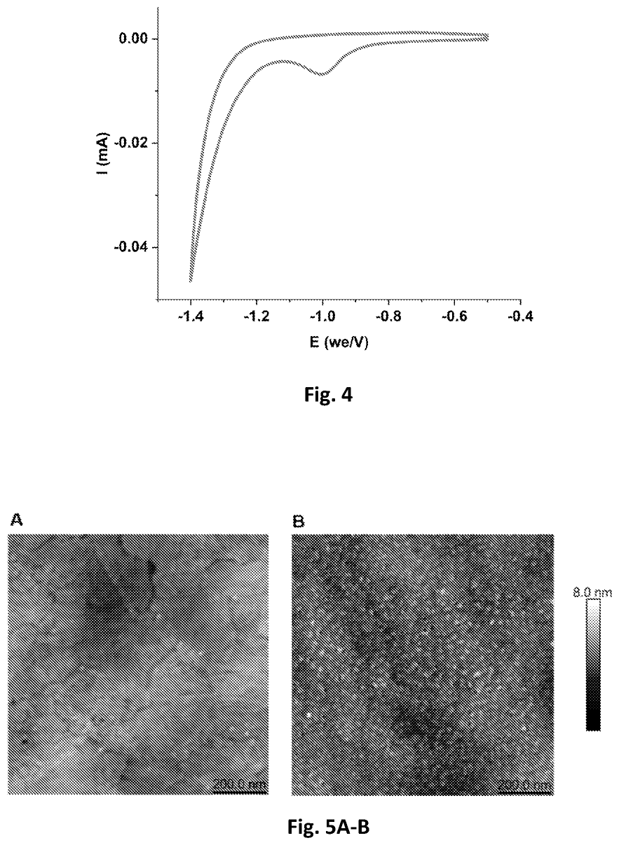

The surface number density (N_{S}) of CFK monolayer was extracted from reductive desorption analysis. The concentration of CFK was calculated by the characteristic peak at −1.1 V (FIG. 4) and yield N_{S}=1.4×10⁻¹⁰ moles CFK/cm². Typical N_{S} of alkanethiol SAM is 9.3×10⁻¹⁰ moles RSH/cm². Our results show that CFK surface coverage is lower than typical alkanethiols assumingly due to repulsion between peptide chains. Since CFK are positively charged molecules with substantial intermolecular electrostatic repulsion, we found our N_{S} comparable to other charged alkanethiol monolayer. The reductive desorption studies support the VASE results, confirming that CFK assemble results in about 10% coverage.

Atomic force microscopy (AFM) analysis was performed to evaluate the topography of CFK peptides monolayer on the gold surface. The results show that while the average surface roughness (Ra) of bare gold is 0.26 nm, the FK monolayer assembly results only in a slight increase to 0.31 nm (FIG. 5). This moderate increase in roughness further hints of the assembly of peptidic-monolayer on the surface.

The surface potential caused by adsorption of CFK monolayer on Au was measured using contact potential difference (CPD). CFK assembly gives ACPD of −0.94(±0.08) V in respect to bare gold substrate. The decrease in the surface potential indicates that the dipole is directed towards the surface. This proves that the surface is covered with positively charged molecules and confirms the presence of lysine rich CFKs peptides.

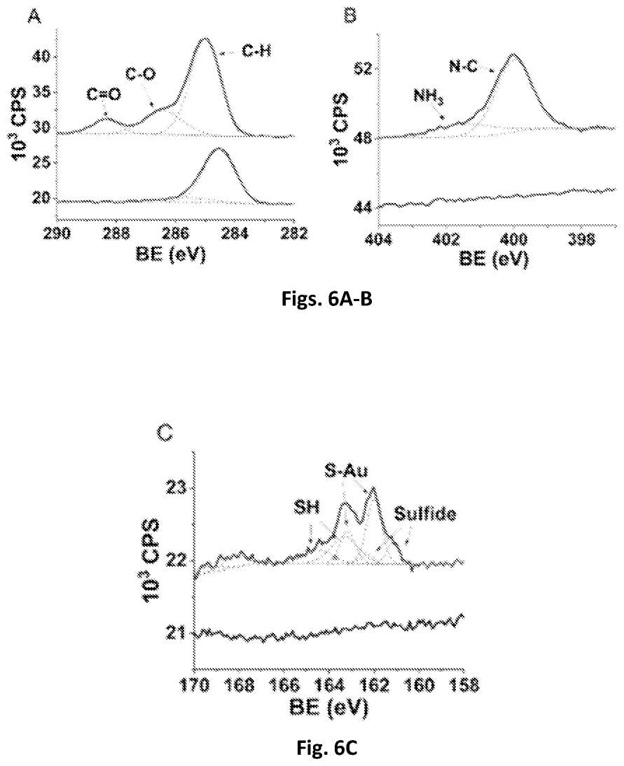

The assembly of CFK peptides on gold surface was also characterized by XPS analysis to trace the peptide at the atomic level features: sulfur, nitrogen and carbon atoms. Binding energy (BE) peak that corresponds with C═O bond of carbonyl at peptide chains was observed at 288.3 eV (FIG. 6A). The two peaks at the nitrogen-related binding energy spectral region, 400 eV and 401.5 eV, correspond with the primary amine and the quaternary amine (FIG. 6B). BE peaks at 162.1 and 163.2 eV correspond with the 2p electrons of the sulfur attached to the gold (FIG. 6C). The peak at 163.4 and 164.6 eV are related to the 2p electrons of unbound thiols (S—H). The ratio between S—Au related peaks and the SH ones (1:1.35) suggests that most CFK is chemisorbed and part of it is physisorbed as π-π interactions between peptide chains or interaction of amines side chains with gold can contribute to the monolayer.

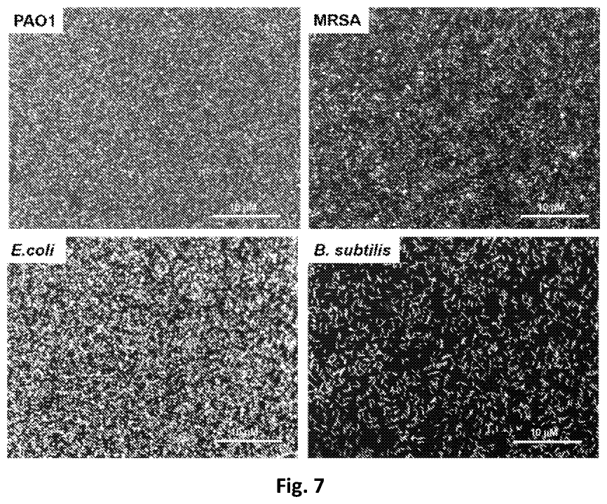

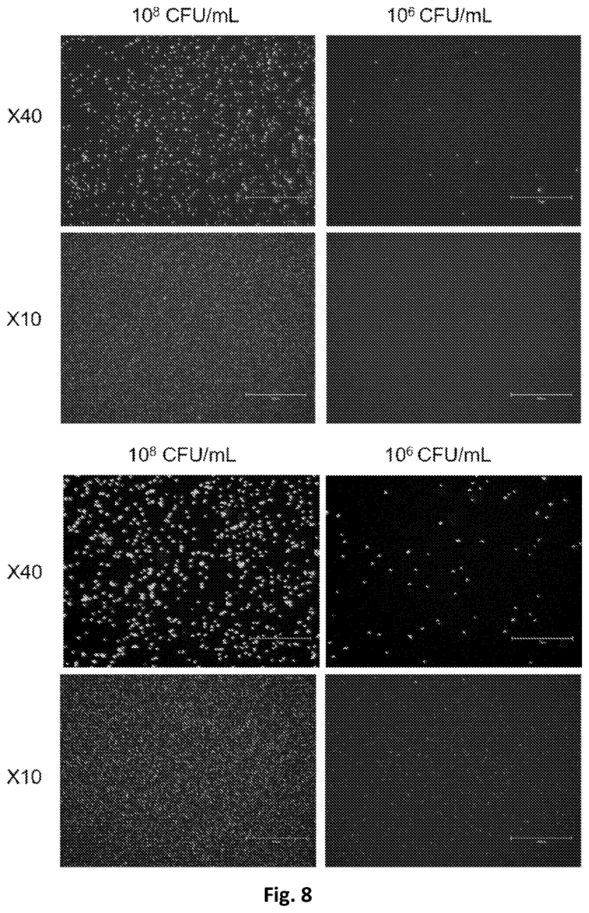

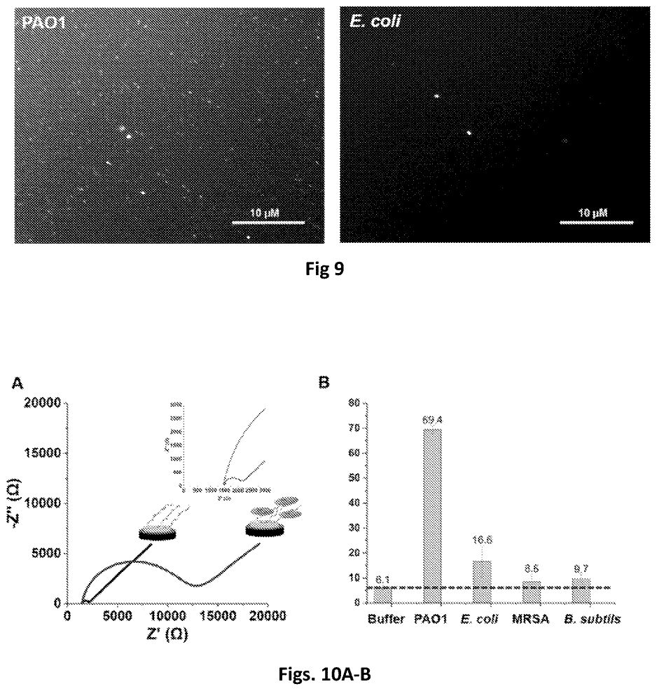

The CFKs monolayers bacteria binding was evaluated. Interaction of FK with bacteria in solution was demonstrated previously. However, when peptides are anchored to a surface their interaction with the bacteria might decrease as their flexibility and the folding to secondary structure can be limited. To study the ability of CFK layer to bind bacterial cells, various bacterial cells both gram positive and gram negative as: (Pseudomonas aeruginosa (PAO1), E. coli, methicillin resistant Staphylococcus aureus (MRSA) and Bacillus subtills) were stained with syto9 fluorescent dye and were incubated on the CFK modified gold surfaces. Binding of all bacterial cells to Au-CFK surfaces was observed at different microbial loads and the maximal coverage was gained at 10⁸ CFU/mL (FIG. 7 and FIG. 8). High coverage of the surface was obtained for PAO1, MRSA and E. coli and lower coverage was obtained for B. subtilis. In a control experiment, the binding of bacteria to bare Au surface was minor (FIG. 9). Au-CFK demonstrate that the anchored peptides maintain their bacterial binding properties.

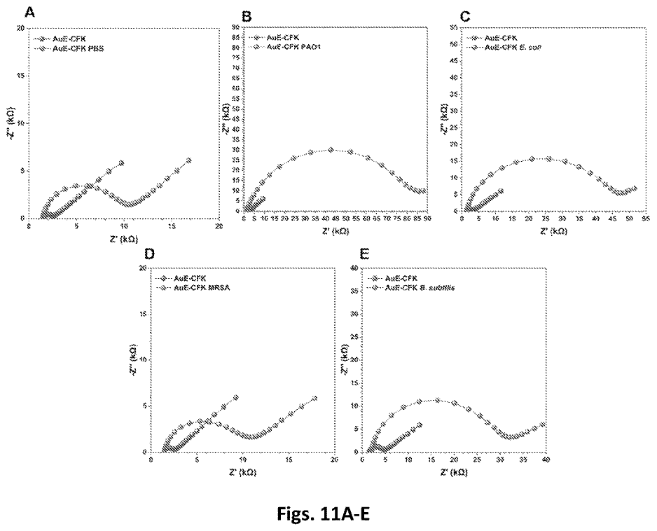

The next step was to develop the sensing platform for detecting bacteria using electrochemistry. EIS were performed prior and after adsorption of CFK to electrodes to form the peptide monolayer of gold electrode (AuE-CFK). The resistance for redox-active [Fe(CN)₆]³⁻/[Fe(CN)₆]⁴⁻ couple penetration (charge transfer resistance, R_{CT}) was fitted according Randles circuit. The R_{CT} values of AuE-CFK was in the range of 600-2000Ω compared to ~80Ω of the bare Au electrode. Subsequently, EIS measurements of AuE-CFK were performed in response to incubation with different bacteria (at the same microbial load). After incubation with bacteria or buffer (negative control) the electrodes were washed with buffer to remove unbound bacteria and measured again. The incubation with 10⁸ CFU/mL E. coli resulted in an R_{CT} increase of ~12,000Ω ((FIG. 10A). AuE-CFK were incubated with three other bacteria and the response is presented in normalized R_{CT} values that was calculated by dividing the measured R_{CT} after exposure to bacteria by the R_{CT} of untreated monolayer measured (FIG. 10B, and FIG. 11). The increase in R_{CT} was bacteria-dependent. PAO1 has the strongest response showing an increase of 69-fold. E. coli presents a 16-fold increase in R_{CT} of while MRSA and B. subtilis both resulted in rather a weak response. Exposure to buffer also causes to increase in R_{CT}. As the CFK monolayer is composed of different sequences, the intramolecular interactions between the positively charged lysine residues and the bulky phosphate-anion of the buffer, the monolayer is densified and exposes a negatively charged interface that repels the negatively charged redox-active couple, ferri-/ferro-cyanide. Therefore, incubation of the layer in buffer cause to re-organization of the layer resulting in a change in the impedimetric signal. However, the response of CFK with PAO1 is an order of magnitude larger than the one observed to the buffer only.

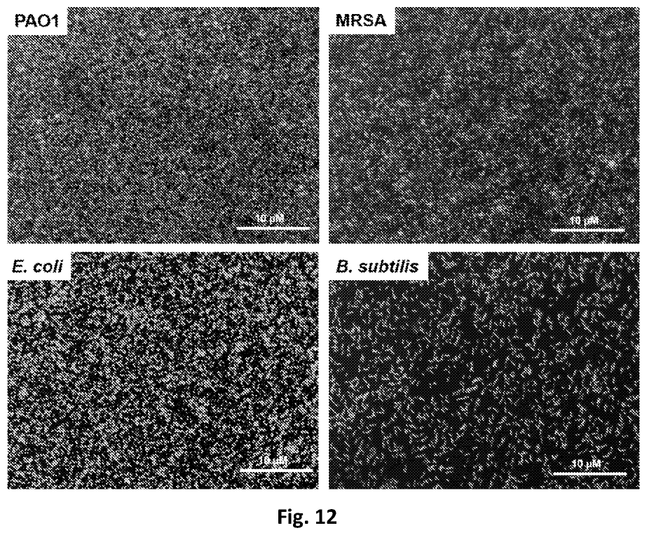

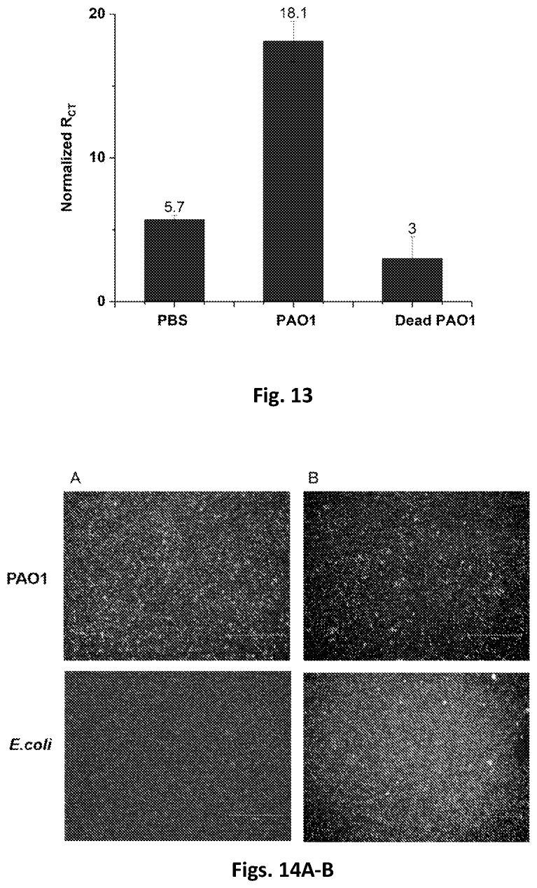

The binding of bacteria to CFK surfaces that was observed by fluorescent microscope did not entirely translate to EIS signal and requires a deeper evaluation of the observed effect. Since the peptides have antimicrobial activity in solution, we checked if the signal related to CFK surfaces was caused by either dead bacteria or the redox active species effect itself. Our control studies showed that dead bacterial cells do not lead to EIS response and that the redox species does not affect the bacteria viability or removal from the surface (FIGS. 12-14).

EIS response results from the permeation of the redox active species through the monolayer hence depends on the changes in the monolayer charge, dipole, density, and morphology which might be affected by the nature of analyte attached to the substrates. The CFK monolayer is positively charged and can interact with [Fe(CN)₆]^{3-/-4} via electrostatic interactions. While bacteria are attached, the positive charge of CFK can be shielded and the changes in the redox species permeation could result in R_{CT} increase.

Gram negative bacteria has more negatively charged surface density than gram positive bacteria. Therefore, interactions with [Fe(CN)₆]^{3-/-4} with PAO1 and E. coli can result in larger electrostatic repulsion in comparison to MRSA and B. subtilis and in higher EIS response. The differences between the response to PAO1 and E. coli are due to the nature of their membranes and other components on the membrane as receptors, saccharides and more. It is known that Zeta potential of PAO1 and E. coli has different values. PAO membrane contains few components that are dominant in effect its surface total charge. Although the main phospholipid of PAO and E. coli is phosphatidylethanolamine that is zwitterionic, PAO has unusual high phosphorous content contribute to its overall negative charge on surface. The LPS of PAO contain special acidic saccharides as 2-keto-3-deoxyoctulosonic (KDO) acid, 2-amino-2-deoxyuronic acids, 2,3-diamino-2,3-dideoxyuronic acids and 5,7-diamino-3,5,7,9-tetradeoxynonulosnic acids. PAO capsule consists of negatively charged o-capsular and alginate polysaccharides. All those components contribute to the negative charge of PAO, the repulsion effect on the redox active species and subsequently provide the basis for the high EIS signal.

### MRSA Specific Binding Peptides

The specific peptides developed herein designated P1-P6 are shown in the Table 1 below:

TABLE 1

| Peptide | Peptide | | Length |
|---|---|---|---|
| No. | name | Sequence | (A.A.) |
| P1 | TST | TSTWNLYVPPEP | 12 |
| PZ | RMY | RMYQSLLAPS | 10 |
| P3 | YSS | YSSATFRLHNIS | 12 |
| P4 | SYA | SYALSSASVRTK | 12 |
| P5 | WPG | WPGPARTQYHRY | 12 |
| P6 | WPG-11A | WPGPARTQYHAY | 12 |

### Immobilizing Peptides on Gold Surfaces

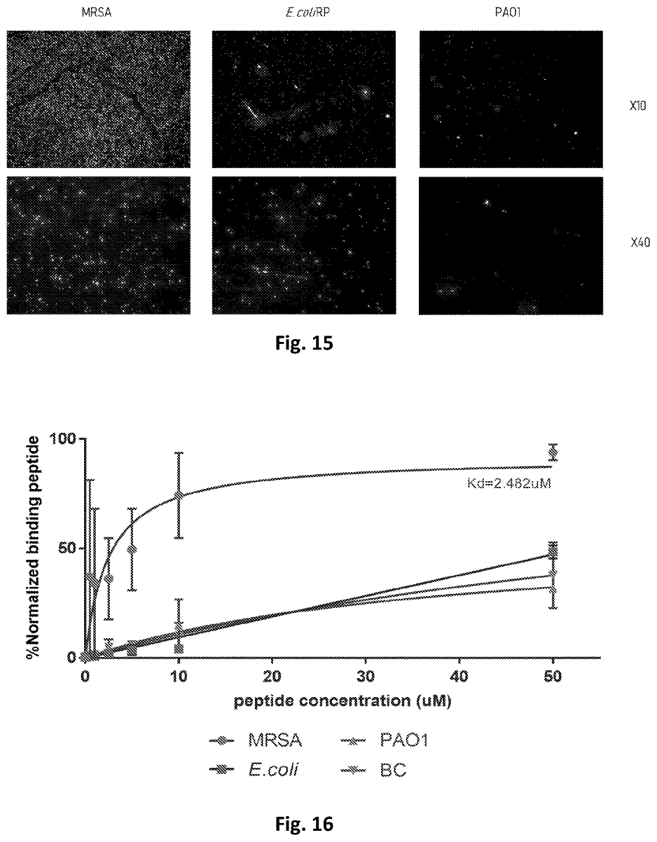

Immobilization of the peptides allows us to examine the affinity of the peptide to the bacteria on a surface. We immobilized the peptides to a gold surface via a thiol-Au bond (FIG. 15). All of the peptides were synthesized with a linker (Gly-Gly-Gly-Cys) at their N′ terminus, so the thiol-Au bond will be formed. MRSA, E. coli RP and PAO1 bacterial cells were grown to a bacterial load of ~10\hat{ }8 CFU/mL (OD595-0.5) in a suspension. The bacterial cells were stained with DAPI (4′,6-diamidino-2-phenylindole), and incubated on the coated gold surface for 10 minutes. The unbounded bacterial cells were then washed three times with PBS and DDW. Next, we observed the surface using EVOS fluorescence microscopy to assess the number of bound bacterial cells on the surface. We compared the binding of different bacterial cells and several derived immobilized peptides.

### Flow Cytometry

Flow cytometry (FC) is a technique used to detect and measure physical and chemical characteristics of a population of cells or particles in a suspension. In our research, we used this method to measure the binding of the labeled peptide binder to the target bacterial cells (FIG. 16). The peptides were labeled with a 5,6-carboxyfluorescein at the N′ terminus. MRSA, E. coli RP and PAO1 bacterial cells were incubated with the labeled peptides. After several cycles of washing the bacterial cells were re-suspended in fresh PBS. The samples were filtered and analyzed by the FC system. FC results were analyzed with FlowJo V10 software in order to quantify the labeled bacterial cells compared to the non-labeled to estimate the ability of the labeled peptide to bind the target bacteria.

### Immobilizing Peptides on Gold Surfaces

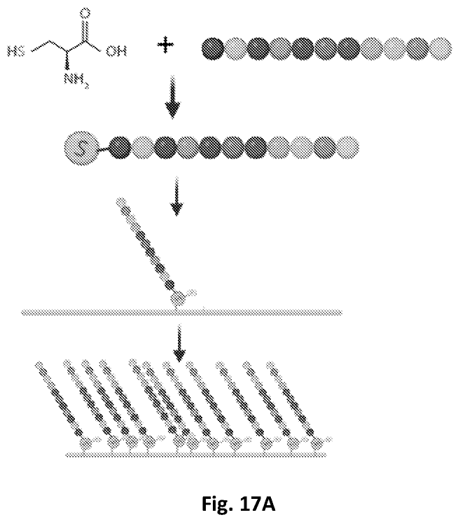

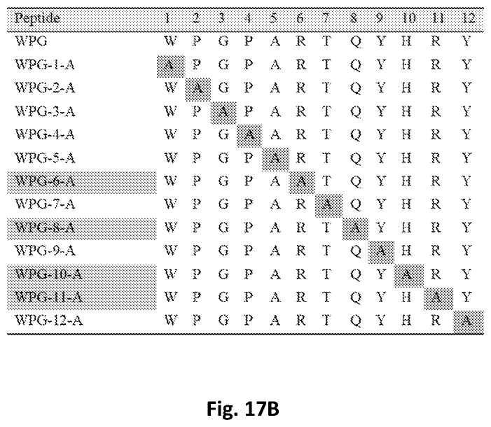

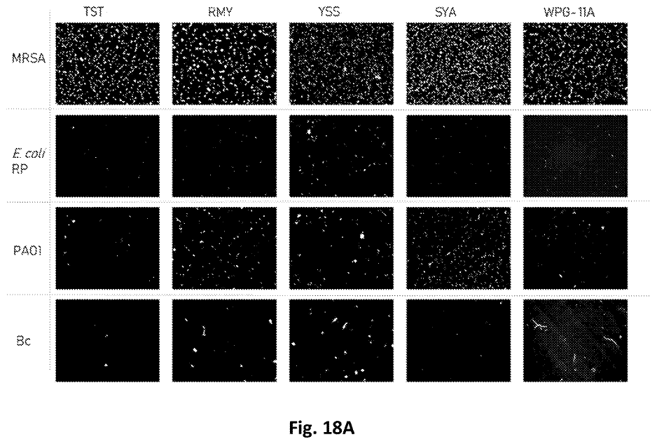

Immobilization of the peptides allows us to examine the affinity of the peptide to the bacteria on a surface. We immobilized the peptides to a gold surface via a thiol-Au bond (FIG. 17A). All of the peptides were synthesized with a linker (Gly-Gly-Gly-Cys) at their N′ terminus, so the thiol-Au bond will be formed.

MRSA, E. coli RP and PAO1 bacterial cells were grown to a bacterial load of ~10\hat{ }8 CFU/mL (OD₅₉₅=0.5) in a suspension. The bacterial cells were stained with DAPI (4′,6-diamidino-2-phenylindole), and incubated on the coated gold surface for 10 minutes. The unbounded bacterial cells were then washed three times with PBS and DDW. Next, we observed the surface using EVOS fluorescence microscopy to assess the number of bounded bacterial cells on the surface. We compared the binding of different bacterial cells and several derived immobilized peptides.

### Characterization and Optimization of the Peptide Sequences

In order to evaluate the role of each amino acid for the bacterial binding, we performed an Alanine scan. In each peptide one amino acid was changed to Alanine (FIG. 17B).

### Results

### Identification of Specific MRSA Peptide Binders Via Phage Display

After the bioinformatics analysis of the next generation sequencing, we selected four different lead peptides. The naïve library of the phages had approximately 10\hat{ }13 PFU/mL different random peptide sequences with different chain lengths (8-12 amino acids), with randomized sequences. Four potential peptides were selected to bind specifically to MRSA bacterial cells and not to other bacterial models (Table 2). In addition to these selected peptides, in our lab we have studied another MRSA peptide binder that was discovered previously (Table 2).

TABLE 2

| Peptide | Peptide | | Length |
|---|---|---|---|
| No. | name | Sequence | (A.A.) |
| P1 | TST | TSTWNLYVPPEP | 12 |
| P2 | RMY | RMYQSLLAPS | 10 |
| P3 | YSS | YSSATFRLHNIS | 12 |
| P4 | SYA | SYALSSASVRTK | 12 |
| P5 | WPG | WPGPARTQYHRY | 12 |

### Peptide-Bacterial Binding Assays

To evaluate the binding affinity and selectivity of the selected peptides to the bacterial cells, we have used two main methods: 1. Flow cytometry to study the binding affinities in solution. 2. Immobilizing the peptides to a gold surface. In both methods, we exposed and incubated the bacteria to the peptides, washed away the unbound peptides, and asses, quantitatively or qualitatively the affinity of the peptide to the tested bacteria. Assessment of the affinity of the peptides toward the bacteria, had been performed in suspension by Flow Cytometry.

### The Peptide Binder WPG and Alanine Scan Derived Peptides

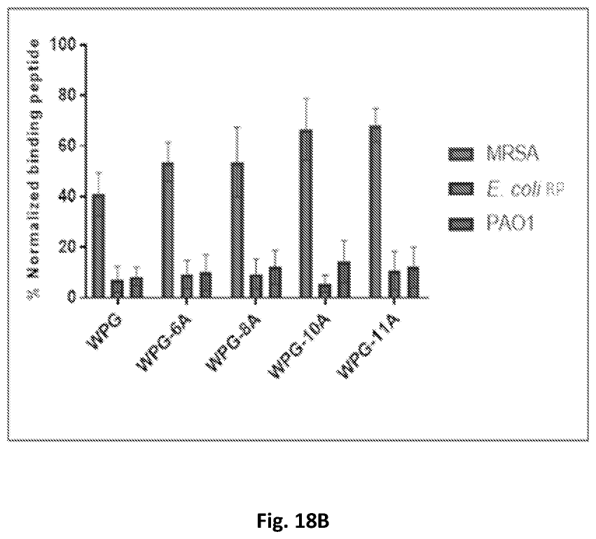

The WPG peptide sequence (P5) has shown high selectivity toward MRSA bacterial cells. It has shown high affinity to MRSA bacterial cells and low binding to the control bacteria (PAO1 and E. coli RP) in a suspension (FC) and also when the peptide was immobilized on a surface (FIG. 18). To further evaluate the peptide binding affinity, we have designed twelve derived peptide sequences, each peptide, with one amino acid that was replaced to Alanine (FIG. 17B). From these twelve options, we chose four peptide sequences (marked in gray, FIG. 17B) to potentially increase it selectivity. We have synthesized and purified those peptides, with (Gly-Gly-Gly-Cys) linker, as described before, and evaluated their peptide binding affinity to MRSA, E. coli RP and PAO1 bacterial cells. As we can see in FIG. 3, the peptide sequence when the 11^{th} amino acid was changed to Alanine (WPG-11A), has shown the highest selectivity, high binding to MRSA and low binding to PAO1 and E. coli RP, in both methods. Also, the WPG-10A peptide sequence has shown higher selectivity then the original WPG peptide sequence, but in comparison to the WPG-11A it was less selective to MRSA cells. Overall, regarding the FC results (FIG. 18B), we can tell that all of the four modified peptide sequences have shown higher binding to MRSA than the original WPG peptide. In contrast, in the immobilized peptide sequences, we can see great improvement with the WPG-11A sequence compared to the other sequences (FIG. 18A).

### Phage Display Peptide Sequences

We have selected the four peptide sequences, (P1-P4, Table 2) via phage display method based on our bioinformatics analysis. These four peptides were synthesized with the (Gly-Gly-Gly-Cys) linker and purified using HPLC. The binding affinity of the peptide to different bacterial cells was evaluated again with FC and by immobilizing the peptides to a gold surface, and compare the result to the WPG peptide sequence. All of the selected peptides have shown high selectivity toward MRSA bacterial cells, in both the tested methods (FIG. 18). These peptides bound strongly MRSA bacterial cells and showed low binding affinities to the other bacterial cells tested. The immobilized TST peptide (P1) has shown the lowest binding of the control bacteria (E. coli RP, PAO1 and Bc), therefore, this peptide was found to be the most selective peptide towards MRSA (FIG. 18A). The YSS peptide (P3) has shown the lowest selectivity when was immobilized on the gold surface, as we observed that all the tested bacteria showed binding to this surface.

### Dead Bacteria Detection:

As FKC are antimicrobial peptides, we examined their effect on killing bacteria when they anchored to a surface. We incubated Au-CFK surfaces with live bacteria stained with live dead dyes for 40 minutes. Using fluorescence microscopy we observed that the death of bacteria was minor. Those results are in correspondence with our previous results (Cheriker et al.) that anchored RPMs delay the antimicrobial activity in comparison to free peptides in solution.

Moreover, we examined the impedimetric sensing of dead bacteria compared to live bacteria. AuE-CFK electrodes were incubated with live bacteria suspension and dead bacteria suspension (killed by addition of CFK). The electrodes were measured before and after incubation with bacteria, Rct was extracted and normalized RCT was calculated. Dead bacteria did not generate a signal in contrast to live bacteria.

### CFK Assembly on a Gold Surface

Gold substrates for surface characterization were prepared by evaporation of 10 nm chrome as adhesion layer followed by evaporation of 100 nm Au on top of highly doped n-type Silicon wafer. The surfaces were washed with ethanol, dried under a mild stream of nitrogen gas, and cleaned using UVOCS (ultraviolet ozone cleaning system) for 20 min.

Solution of 1.4 mg/mL (0.5 mM) CFK was prepared in PBS-L buffer solution (pH=7.4). A variety of buffers was examined. Finally, the adsorption process was completed in PBS buffer with low ionic strength (PBS-L) in order to enable the optimal organization and interaction of the peptides chains. Chemisorption was performed by drop casting CFK solution on bare gold substrates for an overnight incubation (16 h) at 37 °C. The surfaces were washed after incubation with PBS-L buffer and TDW and dried under nitrogen stream. Characterization of CFK-AuAu-CFK surfaces is described at the next section.

### Florescence Microscopy

Fluorescence microscopy was used to evaluate the binding ability of bacteria to CFK-modified Au surfaces. The bacteria cells were grown overnight in LB at 37 °C. The bacterial cells' suspension was diluted 1/50 in fresh LB and grown for additional 3-4 h. Bacteria pellets were washed twice with PBS-L by centrifugation (8000 rpm for 2 min) Bacteria were grown as described above, centrifuged, and washed with PBS-L twice. The bacteria pellet was diluted to OD600 nm=0.5 and stained with Syto9 (live, green; Life Technologies Corp., Eugene, OR, Carlsbad, CA, USA). The suspension was dropped on the immobilized peptide surfaces and incubated for 40 min at resting room temperature. The surfaces were washed with PBS-L and TDW (three times) to remove unbound bacteria and observed with a fully motorized IX81 fluorescent microscope (Olympus, Tokyo, Japan). Images were captured at ×20 magnification in at least three distinct areas of the sample. PMT emission was 490-530 nm.

### Gold Electrodes' Surface Modifications and Impedimetric Sensing

WE were dipped in 0.5 mM (FK)20-Cys in PBS in low ionic strength (PBS-L) containing 10 mM Na₂HPO₄·7H₂O, 1.8 mM KH₂PO₄, 1.37 mM NaCl, and 0.027 mM KCl (pH 7.4) for 16 h at 37 °C, rinsed by dipping in PBS-L, and then measured. Subsequently, the electrodes were exposed to 10⁸ CFU/mL bacterial cells' suspension in PBS-L for 40 min under gentle agitation. The bacteria cells were grown overnight in LB at 37 °C. The bacterial cells' suspension was diluted 1/50 in fresh LB and grown for additional 3-4 h. Bacteria pellets were washed twice with PBS-L by centrifugation (8000 rpm for 2 min) and diluted to an optical density of 0.5 at 600 nm. For dead bacteria suspension, 100 μg/mL FKC were added to kill the PAO1 cells.

Following incubation with bacteria, the electrodes were washed and measured. The EIS characterization was prepared in an EIS solution, containing 1 mM K3[Fe(CN)6] and 1 mM K4[Fe(CN)6] (RedOx species) in PBS-L. Spectra were recorded by applying a single sine AC potential of 10 mV amplitude superimposed with 0.21 V DC potential (vs. RE) and scanning over the frequency range of 100 kHz to 0.1 Hz. The data were analyzed as Nyquist plots and fitted to a Randles-like equivalent circuit of R_S [(R [symbol] __CT W)Q], where RS is the solution resistance, RCT is the interface charge-transfer resistance, W is the Warburg diffusion element, and Q is the constant phase element of non-ideal capacitance. All experiments were completed on at least 3 samples with at least 3 repetitions.

### CONCLUSIONS

We showed a new strategy for bacterial detection that relies on readily accessible RPM as recognition monolayers. Among antimicrobial peptides, CFK can target broad spectrum of bacteria, can be synthetized easily, in short time, and with low cost. Here it is the first time that the co-assembly of random peptides was utilized to form the active layer of electrochemical bacterial biosensor. We showed that those accessible RPM can be assembled on gold surface while maintaining bacteria binding properties. On their immobilized state, CFK mixtures can bind various bacterial cells to the surface. Translation of the binding to electrochemical signal was done by EIS, resulted at highest signal for PAO1 due to its negatively charge extra-cellular components. The study proves that RPM-based biosensors could be used for bacteria detection. Further optimization of device architecture can be used to increase the sensitivity, thus offers a new application for these very intriguing family of synthetic peptides.

1.-95. (canceled)

96. An electrochemical impedimetric recognition surface, comprising a substrate having a surface associated with a monolayer of recognition peptides capable of interacting with a microorganism present in a sample, wherein said interaction between the layer of recognition peptides and the microorganism induces a surface change detectable by electrochemical impedance spectroscopy (EIS), wherein the surface is optionally a surface of an electrode, a sensor or an electrochemical device.

97. The recognition surface according claim 96, wherein the surface is a gold surface or a surface of a glassy carbon electrode (GCE).

98. The recognition surface according to claim 96, wherein each of the recognition peptides comprises a linker moiety of 1 to 5 amino acids.

99. The recognition surface according to claim 96, wherein the monolayer comprises or consists a plurality of recognition peptides, each comprising between 8 and 50 amino acids.

100. The recognition surface according to claim 96, wherein the monolayer comprises or consists a plurality of recognition peptides, each comprising an amino acid sequence being or selected from TSTWNLYVPPEP (SEQ ID NO 2), WPGPARTQYHRY (SEQ ID NO 3), WPGPARTQYHAY (SEQ ID NO 4), RMYQSLLAPS (SEQ ID NO 5), YSSATFRLHNIS (SEQ ID NO 6), and SYALSSASVRTK (SEQ ID NO 7).

101. The recognition surface according to claim 96, wherein the monolayer comprising or consisting a plurality of recognition peptides having sequence TSTWNLYVPPEP-Gly-Gly-Gly-Cys (SEQ ID NO 8); or.
wherein the monolayer comprising or consisting a plurality of recognition peptides having sequence RMYQSLLAPS-Gly-Gly-Gly-Cys (SEQ ID NO 9); or
wherein the monolayer comprising or consisting a plurality of recognition peptides having sequence YSSATFRLHNIS-Gly-Gly-Gly-Cys (SEQ ID NO 10); or
wherein the monolayer comprising or consisting a plurality of recognition peptides having sequence SYALSSASVRTK-Gly-Gly-Gly-Cys (SEQ ID NO 11); or
wherein the monolayer comprising or consisting a plurality of recognition peptides having sequence WPGPARTQYHRY-Gly-Gly-Gly-Cys (SEQ ID NO 12); or
wherein the monolayer comprising or consisting a plurality of recognition peptides having sequence WPGPARTQYHAY-Gly-Gly-Gly-Cys (SEQ ID NO 13); or
wherein the monolayer comprising or consisting a plurality of recognition peptides having sequence Cys-Gly-Gly-Gly-TSTWNLYVPPEP (SEQ ID NO 14); or
wherein the monolayer comprising or consisting a plurality of recognition peptides having sequence Cys-Gly-Gly-Gly-RMYQSLLAPS (SEQ ID NO 15); or
wherein the monolayer comprising or consisting a plurality of recognition peptides having sequence Cys-Gly-Gly-Gly-YSSATFRLHNIS (SEQ ID NO 16); or
wherein the monolayer comprising or consisting a plurality of recognition peptides having sequence Cys-Gly-Gly-Gly-SYALSSASVRTK (SEQ ID NO 17); or
wherein the monolayer comprising or consisting a plurality of recognition peptides having sequence Cys-Gly-Gly-Gly-WPGPARTQYHRY (SEQ ID NO 18); or
wherein the monolayer comprising or consisting a plurality of recognition peptides having sequence Cys-Gly-Gly-Gly-WPGPARTQYHAY (SEQ ID NO 19); or
wherein the monolayer comprising or consisting a plurality of recognition peptides being a random peptide mixture, RPM.

102. An electrode for electrochemical impedance spectroscopy (EIS), the electrode having a recognition surface according to claim 96.

103. An electrochemical impedance spectroscopy (EIS) electrode, the electrode having a gold surface associated with a monolayer of recognition peptide molecules of the form PP-Gly-Gly-Gly-Cys, wherein the association is through the Cys group and wherein PP is a recognition peptide capable of interacting with a microorganism in a liquid medium.

104. A sensor comprising a substrate having a surface-associated with a plurality of recognition peptides, wherein the recognition peptides selected from
(SEQ ID NO 2)
TSTWNLYVPPEP,
(SEQ ID NO 5)
RMYQSLLAPS,
(SEQ ID NO 6)
YSSATFRLHNIS,
(SEQ ID NO 7)
SYALSSASVRTK,
(SEQ ID NO 3)
WPGPARTQYHRY,
and
(SEQ ID NO 4)
WPGPARTQYHAY

105. The sensor according to claim 104, wherein the substrate having a gold surface, or the substrate is a glassy carbon electrode (GCE).

106. An EIS detection device comprising an electrode according to claim 102.

107. The device according to claim 106, for measuring a change in an electrochemical impedance of a surface comprising surface-associated recognition peptides interacting with a microorganism in a sample.

108. The device according to claim 106, for determining presence and/or concentration and/or viability of a microorganism in a sample.

109. The device according to claim 106, wherein the microorganism is a noncellular or unicellular organism.

110. The device according to claim 109, wherein the microorganism is bacteria, gram-negative organisms, gram-positive organisms, yeasts, spores, lichens, fungi, protozoa, virinos, viroids, viruses, phages, and algae.

111. A method for determining at least one microorganism-associated parameter of a microorganism present in a sample, the method comprising contacting an electrochemical impedimetric recognition surface or a device implementing same with a sample containing or suspected of containing the microorganism, wherein the recognition surface comprises a substrate having a surface associated with a layer of recognition peptides capable of interacting with the microorganism present or suspected of being contained in the sample, wherein said interaction between the layer of recognition peptides and the microorganism induces a surface change indicative of said interaction.

112. The method according to claim 111, wherein the liquid sample is a body fluid sample obtained from a subject.

113. The method according to claim 111, for determining an amount or a concentration of a microorganism in a sample, the method comprising contacting said surface with the sample, measuring a change in an impedance signal relative to an impedance signal measured for one or more control samples having known concentrations of the microorganism and determining concentration of the microorganism in the sample.

114. The method according to claim 111, for determining a level or concentration of viable microorganisms in a sample, the method comprising contacting the recognition surface with the sample, measuring a change in an impedance signal relative to an impedance signal measured for one or more control samples having known concentrations of the microorganism and determining a change in the level or concentration of the microorganism in the sample, wherein a reduction in the impedance signal indicates a reduction or an increase in the viable microorganism population in the sample.

115. The method according to claim 111, for determining viability of bacteria in a sample for determining efficiency of a sterilization protocol.
